(19) United States
(12) Patent Application Publication
(10) Pub. No.: US 2025/0213152 A1
(43) Pub. Date: Jul. 3, 2025

(54) BACKGROUND INTERFERENCE MITIGATION FOR HIGH SENSITIVITY KETONE SENSING

(71) Applicant: ABBOTT DIABETES CARE INC., Alameda, CA (US)

(72) Inventors: Cade FOX, Burlingame, CA (US); Stephen OJA, Reno, NV (US); Zenghe LIU, Alameda, CA (US); Tianmei OUYANG, Saratoga, CA (US); Benjamin FELDMAN, Berkeley, CA (US)

(73) Assignee: ABBOTT DIABETES CARE INC., Alameda, CA (US)

(21) Appl. No.: 18/791,037

(22) Filed: Jul. 31, 2024

Related U.S. Application Data
(60) Provisional application No. 63/516,635, filed on Jul. 31, 2023.

(51) Int. Cl. A61B 5/1486; A61B 5/00; A61B 5/145

(52) U.S. Cl. CPC A61B 5/14865; A61B 5/14546; A61B 5/7214

(57) ABSTRACT

The present disclosure relates to a method of improving the sensitivity of sensing ketones that includes providing i) a ketone sensing electrode comprising a ketone-responsive enzyme and a redox mediator; and ii) a background sensing electrode comprising a redox mediator and no ketone-responsive enzyme and applying a potential less than +40 mV to provide a steady state. The ketone sensing electrode and background sensing electrode can be simultaneously or sequentially disconnected from the circuit to allow the charge to accumulate for a set period of time. After sufficient charge has been built up, both electrodes can be reconnected to the circuit. The ketone signal can be measured by subtracting a signal obtained from the background sensing electrode from a signal obtained from the ketone sensing electrode. The present disclosure further relates to a ketone sensor comprising a first sensing electrode that senses ketone and a second sensing electrode that senses the background.

### BACKGROUND

While glucose management is a main focus for diabetes therapy, continuous ketone monitoring can be beneficial for diabetes patients, particularly those with type 1 diabetes. People with type 1 diabetes can experience a potentially deadly complication known as diabetic ketoacidosis (DKA). Continuous ketone sensing has the potential to prevent DKA. Ketone concentration is also correlated with heart failure metrics. For example, patients with elevated ketone concentrations greater than 300 μM are at risk for heart failure. While such measurements are important, in vivo ketone concentrations tend to be relatively low.

Moreover, ascorbate, uric acid, and other compounds naturally found in interstitial fluid generate background interference signal for biosensors, which can reduce analyte measurement accuracy. At ketone concentrations greater than 1 mM, this background interference signal is significantly lower than ketone signal, and ketone concentrations can be accurately determined. However, as ketone concentrations decrease below 1 mM, the ratio of background interference to ketone signal increases, preventing accurate ketone measurements.

Thus, there is a need for a biosensor with improved ketone sensitivity and that can reduce background signal interference signal to provide accurate and continuous ketone monitoring in vivo.

### BRIEF SUMMARY

The present disclosure may provide a method of improving the sensitivity of sensing ketones that includes providing i) a ketone sensing electrode comprising a ketone-responsive enzyme and a redox mediator; and ii) a background sensing electrode comprising a redox mediator and no ketone-responsive enzyme and applying a potential less than +40 mV to provide a steady state. The ketone sensing electrode and background sensing electrode can be simultaneously or sequentially disconnected from the circuit to allow a charge to accumulate for a set period of time. The ketone signal can be measured by subtracting a signal obtained from the background sensing electrode from a signal obtained from the ketone sensing electrode. The present disclosure further may provide a ketone sensor comprising a first sensing electrode that senses ketone and a second sensing electrode that senses the background.

The present disclosure also relates to a method for sensing ketones comprising contacting a biofluid comprising a ketone with:
a) a first sensing electrode comprising a ketone-responsive enzyme and a redox mediator; and
b) a second sensing electrode comprising a redox mediator and no ketone-responsive enzyme;
connecting the first and second sensing electrodes to a circuit and applying a potential less than +40 mV to both electrodes to provide a steady state;
disconnecting the first and second sensing electrodes from the circuit;
accumulating a charge derived from the biofluid contacting the first and second sensing electrodes for a set period of time;
connecting (e.g., reconnecting) the first and second sensing electrodes to the circuit after the set period of time; and
measuring a ketone signal by subtracting a signal obtained from the second sensing electrode from a signal obtained from the first sensing electrode.

The present disclosure also relates to a method for sensing ketones comprising:
(a) contacting first and second sensing electrodes with a biofluid comprising a ketone, wherein the first sensing electrode comprises a ketone-responsive enzyme and a redox mediator and the second sensing electrode comprises a redox mediator and no ketone-responsive enzyme;
(b) connecting the first sensing electrode to a circuit and applying a potential less than +40 mV to provide a steady state;
(c) disconnecting the first sensing electrode from the circuit and connecting the second sensing electrode to the circuit and applying a potential less than +40 mV to provide a steady state;
(d) accumulating a charge derived from the biofluid reacting with the first sensing electrode for a first set period of time;
(e) connecting (e.g., reconnecting) the first sensing electrode to the circuit after the first set period of time and disconnecting the second sensing electrode from the circuit;
(f) accumulating a charge derived from the biofluid reacting with the second sensing electrode for a second set period of time;
(g) connecting (e.g., reconnecting) the second sensing electrode to the circuit after the second set period of time; and
(h) measuring a ketone signal by subtracting a signal obtained from the second sensing electrode from a signal obtained from the first sensing electrode.

In some aspects of this method, the disconnecting and connecting of step (b) and step (c) are simultaneous.

In any of these aspects, the set period of time, the first set period of time, the second set period of time, or any combination thereof is 30 seconds or more.

In any of these aspects, the potential applied is about +5 mV to about −250 mV. In some aspects, the potential applied is about −80 mV.

In any of these aspects, the first sensing electrode comprises a working electrode and a ketone sensing layer on a portion of the working electrode, wherein the ketone sensing layer comprises the ketone-responsive enzyme and the redox mediator.

In any of these aspects, the ketone-responsive enzyme is 3-hydroxybutyrate dehydrogenase.

In any of these aspects, the first sensing electrode further comprises an NAD(P)H oxidoreductase and nicotinamide adenine dinucleotide phosphate (NAD(P)+) or a derivative thereof.

In any of these aspects, the ketone-responsive enzyme is attached to the redox mediator.

In any of these aspects, the first sensing electrode further comprises an albumin.

In any of these aspects, the first sensing electrode further comprises a pH buffer.

In any of these aspects, the second sensing electrode comprises a working electrode and a background sensing layer on a portion of the working electrode, wherein the background sensing layer comprises a redox mediator. In an aspect, the redox mediator in the first and second sensing electrodes are the same material.

In any of these aspects, the redox mediator comprises a polymer and an electron transfer agent. In some aspects, the polymer comprises poly(vinylpyridine), poly(thiophene), poly(aniline), poly(pyrrole), or poly(acetylene). In some aspects, the polymer comprises a polymer or copolymer repeat unit comprising at least one pendant pyridinyl group, imidazolyl group, or both a pyridinyl and imidazolyl group. In some aspects, the electron transfer agent comprises a transition metal complex. In some aspects, the transition metal complex comprises osmium, ruthenium, iron, cobalt, or a combination thereof. In some aspects, the transition metal complex is an osmium transition metal complex comprising one or more ligands, wherein at least one ligand comprises a nitrogen-containing heterocycle.

In any of these aspects, the redox mediator comprises an osmium complex bonded to a poly(vinylpyridine)-based polymer. In some aspects, the polymer is crosslinked with a cross linking agent. In some aspects, the cross linking agent is a polyepoxide, cyanuric chloride, N-hydroxysuccinimide, an imidoester, epichlorohydrin, or a combination thereof. In some aspects, the cross linking agent is a polyethylene glycol diglycidylether (PEGDGE).

In some of these aspects, the ketone sensing layer or the background sensing layer is continuous, or both sensing layers are continuous. In some aspects, the ketone sensing layer or the background sensing layer is discontinuous, or both sensing layers are discontinuous.

In some aspects, a membrane overcoats at least the ketone sensing layer, at least the background sensing layer, or both. In some aspects, the membrane comprises poly(4-vinyl pyridine).

In any of these aspects, the first and second sensing electrodes are part of a sensor comprising a housing. In some aspects, the sensor further comprises a sensor tail configured for implantation into a tissue, wherein the first and second sensing electrodes are disposed on the sensor tail. In some aspects, the sensor further comprises a reference electrode, a counter electrode, or both a reference electrode and a counter electrode. In some aspects, the sensor further comprising at least one insulation layer. In some aspects, the sensor further comprise at least one substrate, wherein the first sensing electrode or the second sensing electrode is disposed on the substrate, or both sensing electrodes are disposed on the substrate.

The present disclosure further relates to a ketone sensor comprising a first sensing electrode comprising a first working electrode and a ketone sensing layer on a portion of the first working electrode, wherein the ketone sensing layer comprises a ketone-responsive enzyme and a redox mediator; and a second sensing electrode comprising a second working electrode and a background sensing layer on a portion of the second working electrode, wherein the background sensing layer comprises a redox mediator and no ketone-responsive enzyme.

In some aspects, the ketone-responsive enzyme is 3-hydroxybutyrate dehydrogenase. In some aspects, the first sensing electrode further comprises an NAD(P)H oxidoreductase and nicotinamide adenine dinucleotide phosphate (NAD(P)+) or a derivative thereof. In some aspects, the first sensing electrode further comprises an albumin. In some aspects, the first sensing electrode further comprises a pH buffer.

In some aspects, the ketone-responsive enzyme is attached to the redox mediator.

In an aspect, the redox mediator in the first and second sensing electrodes are the same material. In some aspects, the redox mediator comprises a polymer and an electron transfer agent.

In some aspects, the polymer comprises poly(vinylpyridine), poly(thiophene), poly(aniline), poly(pyrrole), or poly(acetylene). In some aspects, the polymer comprises a polymer or copolymer repeat unit comprising at least one pendant pyridinyl group, imidazolyl group, or both a pyridinyl and imidazolyl group. In some aspects, the polymer is crosslinked with a cross linking agent. In some aspects, the cross linking agent is a polyepoxide, cyanuric chloride, N-hydroxysuccinimide, an imidoester, epichlorohydrin, or a combination thereof. In some aspects, the cross linking agent is a polyethylene glycol diglycidylether (PEGDGE).

In some aspects, the electron transfer agent comprises a transition metal complex. In some aspects, the transition metal complex comprises osmium, ruthenium, iron, cobalt, or a combination thereof. In some aspects, the transition metal complex is an osmium transition metal complex comprising one or more ligands, wherein at least one ligand comprises a nitrogen-containing heterocycle. In some aspects, the redox mediator comprises an osmium complex bonded to a poly(vinylpyridine)-based polymer.

In some aspects, the ketone sensing layer or the background sensing layer is continuous, or both sensing layers are continuous on the working electrode. In some aspects, the ketone sensing layer or the background sensing layer is discontinuous, or both sensing layers are discontinuous on the working electrode.

In some aspects, the sensor further comprises a membrane overcoating at least the ketone sensing layer, at least the background sensing layer, or both. In some aspects, the membrane comprises poly(4-vinyl pyridine).

In some aspects, the sensor further comprises a housing. In some aspects, the sensor further comprises a sensor tail configured for implantation into a tissue, wherein the first and second sensing electrodes are disposed on the sensor tail. In some aspects, the sensor further comprises a reference electrode, a counter electrode, or both a reference electrode and a counter electrode. In some aspects, the sensor further comprises at least one insulation layer. In some aspects, the sensor further comprises at least one substrate, wherein the first sensing electrode or the second sensing electrode is disposed on the substrate, or both sensing electrodes are disposed on the substrate.

Additional aspects and advantages of the disclosure will be set forth, in part, in the description that follows, and will flow from the description, or can be learned by practice of the disclosure.

It is to be understood that both the foregoing summary and the following detailed description are exemplary and explanatory only, and do not restrict the scope of the claims.

### BRIEF DESCRIPTION OF THE DRAWINGS/FIGURES

FIG. 1 shows a diagram of an illustrative sensing system that can incorporate an analyte sensor of the present disclosure.

FIGS. 2A-2C show cross-sectional diagrams of analyte sensors including a single sensing layer.

FIGS. 3A-3C show cross-sectional diagrams of analyte sensors including two sensing layers.

FIG. 4 shows a cross-sectional diagram of an analyte sensor including two sensing layers.

FIGS. 5A-5C show perspective views of analyte sensors including two sensing layers upon separate working electrodes.

FIG. 6 shows a sensor current (nA) versus time (hours) plot of a Blank (background) sensor and Standard (ketone) sensor with different sensing potentials applied: +40 mV or −80 mV, each relative to an Ag/AgCl reference.

FIG. 7 shows a sensor current (nA) versus time (hours) plot of a Blank (background) sensor and Standard (ketone) sensor at −80 mV vs. Ag/AgCl reference. The taller double headed arrow is the Standard baseline for 190 μM ketone in serum+background signal. The smaller double headed arrow is the Blank baseline for background signal only.

FIG. 8A show a sensor current (nA) versus time (hours) plot of an exemplary ketone sensor of the present disclosure using accumulation mode sensing. FIG. 8B shows the integrated charge for each peak with the taller double headed arrow indicating the Standard baseline for 190 μM ketone in serum+background signal, and the smaller double headed arrow indicating the Blank baseline for background signal only.

FIG. 9A show a sensor current (nA) versus time (hours) plot of an exemplary ketone sensor (“Sensor 1”) of the present disclosure using cumulative detection and a second ketone sensor (“Sensor 2”) that measured ketone concentration using standard amperometry at −80 mV vs Ag/AgCl. FIG. 9B shows the sensor current (nA) versus time (hours) plot from a time window of 4:00 PM to 12:00 AM.

FIG. 10 shows a sensor current (nA) versus time (hours) plot of an exemplary ketone sensor (Sensor 1) of the present disclosure after background subtraction.

FIG. 11A shows a sensor current (nA) versus time (hours) plot of exemplary ketone sensors (Sensor s1 and 2) of the present disclosure using cumulative detection. FIG. 11B shows a sensor current (up to 10 nA) versus time (hours) plot of exemplary ketone sensors (Sensors 1 and 2) of the present disclosure using cumulative detection.

FIG. 12 shows a sensor current (nA) versus time (hours) plot of (1) exemplary ketone sensors (Sensors 1 and 2) of the present disclosure after background subtraction; and (2) blood ketone test strip measurements.

### DETAILED DESCRIPTION

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular aspects, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a,” “an,” and “the” are used herein to refer to one or to more than one (i.e., to at least one) of the grammatical object of the article. By way of example, “an element” means one element or more than one element.

As used herein, the term “about” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 10% (e.g., up to 5% or up to 1%) of a given value.

The term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given aspect disclosed herein “comprises” certain elements, it should be understood that present disclosure also specifically contemplates and discloses aspects that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an aspect are included.

As used herein, the terms “consists of,” “consisting of,” and the like are to be construed as closed terms, such that an aspect “consisting of” a particular set of elements excludes any element, step, or ingredient not specified in the aspect.

As used herein, “a set period of time” is the amount of time needed to perform a particular step, e.g., contacting a biofluid to a sensing electrode, contacting one or more sensing electrode to a circuit, or accumulating an electronic charge to provide a sufficient signal output that can be measured and quantified for a given analyte (e.g., a ketone). The various set periods of time described herein can be the same or different. Typically, and as discussed in detail elsewhere herein, the set period of time can be about 1 second or more (e.g., about 5 seconds or more, about 10 seconds or more, or about 30 seconds or more) and about 30 minutes or less (e.g., about 20 minutes or less, about 10 minutes or less, about 5 minutes or less, about 3 minutes or less, or about 1 minute or less).

As used herein, the phrase “accumulation mode sensing” refers to the accumulation of electrons produced from the oxidation of an analyte, the oxidation occurring at or on the sensing element of a working electrode that is not connected to a circuit, thereby creating the accumulation of electrons.

As used herein, an “analyte” is an enzyme substrate that is subject to be measured or detected. The analyte can be from, for example, a biofluid and can be tested in vivo, ex vivo, or in vitro. In most aspects herein, the analyte is ketone.

As used herein, the term “background interferents” or “interferents” are substances in an assayed sample that can prevent a desired analyte (e.g., ketone) from being accurately measured. Common interferents include, e.g., ascorbic acid, uric acid, homovanillic acid, 5-hydroxy-tryptamine, catecholamines (e.g., dopamine, noradrenaline and their major metabolites, such as 3,4-dihydroxyphenylacetic acid, 3-methoxytyramine), indolamines, drug metabolites, fibrinogen, proteins, cells (e.g., white blood cells, red blood cells), metal ions (e.g., copper ions, mercury ions), and combinations thereof.

As used herein, a “biofluid” is any bodily fluid or bodily fluid derivative in which the analyte can be measured. Examples of biofluid include, for example, dermal fluid, subcutaneous fluid, interstitial fluid, plasma, blood (e.g., from a vein or blood vessel), lymph, synovial fluid, cerebrospinal fluid, saliva, bronchoalveolar lavage, amniotic fluid, sweat, or tears. In certain aspects, the biological fluid is dermal fluid or interstitial fluid.

As used herein, the phrase “configured to accumulate charge” is an arrangement of the working electrode and circuit that allows for the accumulation of electrons produced from the oxidation of the analyte (e.g., ketone). The oxidation occurring at or on the sensing element of the working electrode is not connected to the circuit, thereby creating an accumulation of electrons.

As used herein, the term “counter electrode” refers to an electrode paired with the working electrode, through which passes a current equal in magnitude and opposite in sign to the current passing through the working electrode. In the context of aspects of the present disclosure, the term “counter electrode” includes both a) counter electrodes and b) counter electrodes that also function as reference electrodes (i.e., counter/reference electrodes), unless otherwise indicated.

As used herein, the term “crosslinking agent” is a molecule that contains at least two (e.g., 2, 3, or 4) reactive groups (e.g., terminal functional groups) that can link at least two molecules together (intermolecular crosslinking) or at least two portions of the same molecule together (intramolecular crosslinking). A crosslinking agent having more than two reactive groups can be capable of both intermolecular and intramolecular crosslinkings at the same time.

As used herein, the term “electrolysis” refers to the electrooxidation or electroreduction of a compound either directly at an electrode or via one or more electron transfer agents.

As used herein, the term “electron transfer agent” refers to a compound that carries electrons between the analyte and the working electrode, either directly, or in cooperation with other electron transfer agents. One example of an electron transfer agent is a redox mediator.

As used herein, components are “immobilized” or “attached” to a polymer and/or a sensor, for example, when the components are entrapped on, entrapped within, covalently bound, ionically bound, electrostatically bound, or coordinatively bound to constituents of a polymer, a sol-gel matric, membrane, and/or sensor, which reduces or precludes mobility.

As used herein, the term “non-leachable” compound, or a compound that is “non-leachably disposed” is meant to define a compound that is affixed on the sensor such that it does not substantially diffuse away from the sensing layer of the working electrode for the period in which the sensor is used (e.g., the period in which the sensor is implanted in a patient or measuring a sample).

As used herein, the term “patient” refers to a living animal, and thus encompasses a living mammal and a living human, for example. The term “user” can be used herein as a term that encompasses the term “patient.”

As used herein, the term “precursor polymer” refers to the starting polymer before the various modifier groups are attached to form a modified polymer.

As used herein, the term “reactive group” refers to a functional group of a molecule (e.g., a polymer, a crosslinking agent, an enzyme) that is can react with another compound to couple at least a portion (e.g., another reactive group) of that other compound to the molecule. Reactive groups include carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups. Activated esters, as understood in the art, generally include esters of succinimidyl, benzotriazolyl, or aryl substituted by electron-withdrawing groups such as sulfo, nitro, cyano, or halo groups; or carboxylic acids activated by carbodiimides.

As used herein, the term “redox mediator” refers to an electron-transfer agent for carrying electrons between an analyte, an analyte-reduced or analyte-oxidized, enzyme, and an electrode, either directly, or via one or more additional electron-transfer agents. A redox mediator that includes a polymeric backbone can also be referred to as a “redox polymer.”

As used herein, the term “reference electrode” includes both a) reference electrodes and b) reference electrodes that also function as counter electrodes (i.e., counter/reference electrodes), unless otherwise indicated.

As used herein, the term “sensing layer” refers to a component of the sensor including constituents that facilitate the electrolysis of the analyte. The sensing layer can include constituents such as a redox mediator (e.g., an electron transfer agent or a redox polymer), a catalyst (e.g., an analyte-specific enzyme), which catalyzes a reaction of the analyte to produce a response at the working electrode, or both an electron transfer agent and a catalyst. In some aspects of the present disclosure, a sensor includes a sensing layer that is non-leachably disposed in proximity to or on the working electrode.

As used herein, the term “sensing element” refers to an application or region of an analyte-specific enzyme disposed with the sensing layer. As such, a sensing element is capable of interacting with the analyte. A sensing layer can have more than one sensing element making up the analyte detection area disposed on the working electrode. In some aspects, the sensing element includes an analyte-specific enzyme and an electron transfer agent (e.g., electron transfer agent). In some aspects, the sensing element includes an analyte specific enzyme, a redox mediator, and a crosslinking agent.

As used herein, the term “sensor” refers to a device configured to detect the presence and/or measure the level of an analyte in a sample via electrochemical oxidation and reduction reactions on the sensor. These reactions are transduced to an electrical signal that can be correlated to (e.g., is proportional to) an amount, concentration, or level of an analyte in the sample.

As used herein, the term “continuous” as it relates to a continuous analyte sensor (e.g. “a continuous ketone sensor”) refers to a sensor that is configured to take one or more measurements of the analyte (e.g. ketone) over a period of time. A continuous sensor may take sequential measurements according to its sampling frequency. For example, one or more measurements may be taken about every 1 ms, about every 10 ms, about every 100 ms, about every 1 s, about every 10 seconds, about every 30 seconds, about every minute, about every 5 minutes, about every 10 minutes, about every 30 minutes, or about every hour. The measurements may be taken continuously e.g. over a contiguous time period of at least 1 hour, 6 hours, 12 hours, 1 day, 2 days, 3 days, 4 days, 5 days, 6 days, 1 week, 2 weeks, 3 weeks, 1 month or longer. A continuous ketone sensor is typically continuously in contact with a sample, such as a biofluid. For example, a continuous ketone sensor may comprise an implantable portion or member as defined herein which in use is in continuous contact with a biofluid such as dermal fluid or interstitial fluid, such that measurements can be taken continuously or periodically according to the sampling frequency of the sensor over the continuous time period.

As used herein, the term “substituted” functional group (e.g., substituted alkyl, alkenyl, alkoxy, aryl) includes at least one substituent (e.g., 1, 2, 3, 4, or 5) that can be, for example, halo, alkoxy, mercapto, aryl, alkoxycarbonyl, alkylaminocarbonyl, dialkylaminocarbonyl, hydroxy, amino, alkylamino, dialkylamino, trialkylammonium, alkanoylamino, arylcarboxamido, hydrazino, alkylthio, alkenyl, and reactive groups.

As used herein, the term “working electrode” refers to an electrode at which the analyte or background interferent is electrooxidized or electroreduced with or without the agency of an electron transfer agent.

As used herein, the term “C₆₋₃₀ aryl” refers to an aromatic compound comprising a mono-, bi-, or tricyclic carbocyclic ring system having one, two, or three aromatic rings, for example, phenyl, naphthyl, anthracenyl, or biphenyl. The aromatic compound generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2 π electrons, according to Hückel's Rule, wherein n=1, 2, or 3.

As used herein, the term “halo” refers to a radical of a halogen, i.e., F, Cl, Br, or I.

As used herein, the term “C₁₋₆ alkyl” refers to a straight-chain or branched alkyl substituent containing from, for example, from about 1 to about 6 carbon atoms, e.g., from about 1 to about 4 carbon atoms or about 1 to about 3 carbons. Examples of alkyl group include, but are not limited to, methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., C₁₋₆ haloalkyl (e.g., -trifluoromethyl (—CF₃)).

As used herein, the term “C₂₋₆ alkenyl” refers to a linear alkenyl substituent containing from, for example, 2 to about 6 carbon atoms (branched alkenyls are about 3 to about 6 carbons atoms). In accordance with an aspect, the alkenyl group is a C₂₋₄ alkenyl. Examples of alkenyl group include, but are not limited to, ethenyl, allyl, 2-propenyl, 1-butenyl, 2-butenyl, 1-pentenyl, 2-pentenyl, 3-pentenyl, 1-hexenyl, and the like.

As used herein, the term “C₂₋₆ alkynyl” refers to a linear alkynyl substituent containing from, for example, 2 to about 6 carbon atoms (branched alkynyls are about 3 to about 6 carbons atoms). In accordance with an aspect, the alkynyl group is a C₂₋₄ alkynyl. Examples of alkynyl group include, but are not limited to, ethynyl, propynyl, 1-butynyl, 2-butynyl, 1-pentynyl, 2-pentynyl, 3-pentynyl, 1-hexynyl, and the like.

As used herein, the term “hydroxy” refers to —OH.

As used herein, the term “nitro” refers to —NO₂.

As used herein, the term “cyano” refers to —CN.

As used herein, the term “amino” refers to —NH₂. The terms mono- and di-C₁₋₆ alkylamino refer to a nitrogen bonded to one or two C₁₋₆ alkyl groups, respectively, i.e., —NHR or —NRR′, in which R and R′ are the same or different C₁₋₆ alkyl groups.

As used herein, the term “C₁₋₆ alkoxy” refers to a C₁₋₆ alkyl group bonded to an oxygen, i.e., —OR, in which R is a C₁₋₆ alkyl group.

As used herein, the term “C₆₋₁₀ aryloxy” refers to an aryl group bonded to an oxygen, i.e., —O(Ar), in which Ar is a C₆₋₁₀ aryl group.

As used herein, the term “aralkoxy” refers to the group —OR(Ar), in which R is a C₁₋₆ alkyl group and Ar is a C₆₋₁₀ aryl group.

As used herein, the term “carboxy” refers to —C(O)OH.

As used herein, the term “C₁₋₆ alkylcarboxy” refers to a carboxy group wherein the hydrogen bound to the carboxy group has been replaced with a C₁₋₆ alkyl group, i.e., —C(O)OR, wherein R is a C₁₋₆ alkyl group.

As used herein, the term “amido” refers to the structure —C(O)NH or —NHC(O). The term “C₁₋₆ alkylamido” refers to —C(O)NR or —NRC(O), wherein R is C₁₋₆ alkyl.

As used herein, the term “C₁₋₆ haloalkylamido” refers to a C₁₋₆ alkylamido group in which the C₁₋₆ alkyl group is substituted with 1, 2, or 3 halo groups, as described herein.

As used herein, the term “heteroaryl” refers to an aromatic compound, as described herein, containing a 5 or 6 membered ring in which 1 or 2 carbons have been replaced with nitrogen, sulfur, and/or oxygen. Examples of heteroaryl include, but are not limited to, pyridinyl, furanyl, pyrrolyl, quinolinyl, thiophenyl, indolyl, oxazolyl, isoxazolyl, pyrazolyl, imidazolyl, thiazolyl, isothiazolyl, 1,3,4-thiadiazolyl, pyridazinyl, pyrimidinyl, pyrazinyl, and triazinyl.

As used herein, the term “heterocycloalkyl” refers to a monocyclic, bicyclic, or spiro ring system containing 3 to 7 carbon atom ring members and 1, 2, or 3 other atoms selected from nitrogen, sulfur, and/or oxygen. Examples of such heterocycloalkyl rings include, but are not limited to, aziridinyl, oxiranyl, thiazolinyl, imidazolidinyl, piperazinyl, homopiperazinyl, pyrrolinyl, pyrrolidinyl, tetrahydrofuranyl, tetrahydrothiofuranyl, pyranyl, tetrahydropyranyl, piperidinyl, and morpholinyl.

### Methods, Sensors, and Compositions of the Disclosure

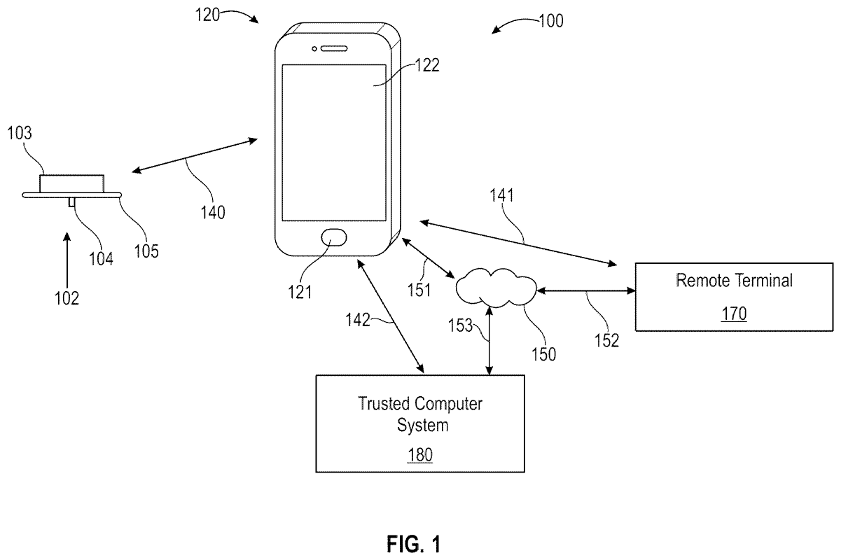

Before describing the analyte sensors of the present disclosure and their components in further detail, a brief overview of suitable in vivo analyte sensor configurations and sensor systems employing the analyte sensors will be provided so that the aspects of the present disclosure can be better understood. FIG. 1 shows a diagram of an illustrative sensing system that can incorporate an analyte sensor of the present disclosure. As shown, sensing system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing analyte concentrations and alerts or notifications determined by sensor 104 or a processor associated therewith, as well as allowing for one or more user inputs, according to certain aspects. Reader device 120 can be a multi-purpose smartphone or a dedicated electronic reader instrument. While only one reader device 120 is shown, multiple reader devices 120 can be present in certain instances. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer system 180 without an intervening reader device 120 being present. For example, but not by the way of limitation, sensor 104 can communicate with remote terminal 170 and/or trusted computer system 180 through a direct communication link to network 150, according to certain aspects, as described in U.S. Patent Application Publication 2011/0213225 and incorporated herein by reference in its entirety. Any suitable electronic communication protocol can be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 can be accessible, according to certain aspects, by individuals other than a primary user who have an interest in the user's analyte levels. Reader device 120 can include display 122 and optional input component 121. Display 122 can include a touch-screen interface, according to certain aspects.

Sensor control device 102 includes sensor housing 103, which can house circuitry and a power source for operating sensor 104. Optionally, the power source and/or active circuitry can be omitted. A processor (not shown) can be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to certain aspects.

Sensor 104 is adapted to be at least partially inserted into a tissue of interest, such as within the dermal or subcutaneous layer of the skin. Sensor 104 can include a sensor tail of sufficient length for insertion to a desired depth in a given tissue. The sensor tail can include at least one working electrode. In certain configurations, the sensor tail can include a sensing layer for detecting an analyte (e.g., ketone). A counter electrode can be present in combination with the at least one working electrode. Particular electrode configurations upon the sensor tail are described in more detail below.

The sensing layer can be configured for detecting a particular analyte (e.g., ketone). For example, but not by way of limitation, the disclosed analyte sensors include at least one sensing layer configured to detect an analyte (e.g., ketone).

In certain aspects of the present disclosure, an analytes (e.g., ketone) can be monitored in any biological fluid of interest such as dermal fluid, interstitial fluid, plasma, blood, lymph, synovial fluid, cerebrospinal fluid, saliva, bronchoalveolar lavage, amniotic fluid, or the like. In certain particular aspects, analyte sensors of the present disclosure can be adapted for assaying dermal fluid or interstitial fluid to determine a concentration of one or more analytes in vivo. In certain aspects, the biological fluid is interstitial fluid.

Referring still to FIG. 1, sensor 104 can automatically forward data to reader device 120. For example but not by the way of limitation, analyte concentration data (i.e., glucose concentration) can be communicated automatically and periodically, such as at a certain frequency as data is obtained or after a certain time period has passed, with the data being stored in a memory until transmittal (e.g., every minute, five minutes, or other predetermined time period). In certain other aspects, sensor 104 can communicate with reader device 120 in a non-automatic manner and not according to a set schedule. For example, but not by the way of limitation, data can be communicated from sensor 104 using RFID technology when the sensor electronics are brought into communication range of reader device 120. Until communicated to reader device 120, data can remain stored in a memory of sensor 104. Thus, a user does not have to maintain close proximity to reader device 120 at all times, and can instead upload data at a convenient time. In certain other aspects, a combination of automatic and non-automatic data transfer can be implemented. For example, and not by the way of limitation, data transfer can continue on an automatic basis until reader device 120 is no longer in communication range of sensor 104.

An introducer can be present transiently to promote introduction of sensor 104 into a tissue. In certain illustrative aspects, the introducer can include a needle or similar sharp. As would be readily recognized by a person skilled in the art, other types of introducers, such as sheaths or blades, can be present in alternative aspects. More specifically, the needle or other introducer can transiently reside in proximity to sensor 104 prior to tissue insertion and then be withdrawn afterward. While present, the needle or other introducer can facilitate insertion of sensor 104 into a tissue by opening an access pathway for sensor 104 to follow. For example, and not by the way of limitation, the needle can facilitate penetration of the epidermis as an access pathway to the dermis to allow implantation of sensor 104 to take place, according to one or more aspects. After opening the access pathway, the needle or other introducer can be withdrawn so that it does not represent a sharps hazard. In certain aspects, suitable needles can be solid or hollow, beveled or non-beveled, and/or circular or non-circular in cross-section. In more particular aspects, suitable needles can be comparable in cross-sectional diameter and/or tip design to an acupuncture needle, which can have a cross-sectional diameter of about 250 microns. However, suitable needles can have a larger or smaller cross-sectional diameter if needed for certain particular applications.

In certain aspects, a tip of the needle (while present) can be angled over the terminus of sensor 104, such that the needle penetrates a tissue first and opens an access pathway for sensor 104. In certain aspects, sensor 104 can reside within a lumen or groove of the needle, with the needle similarly opening an access pathway for sensor 104. In either case, the needle is subsequently withdrawn after facilitating sensor insertion.

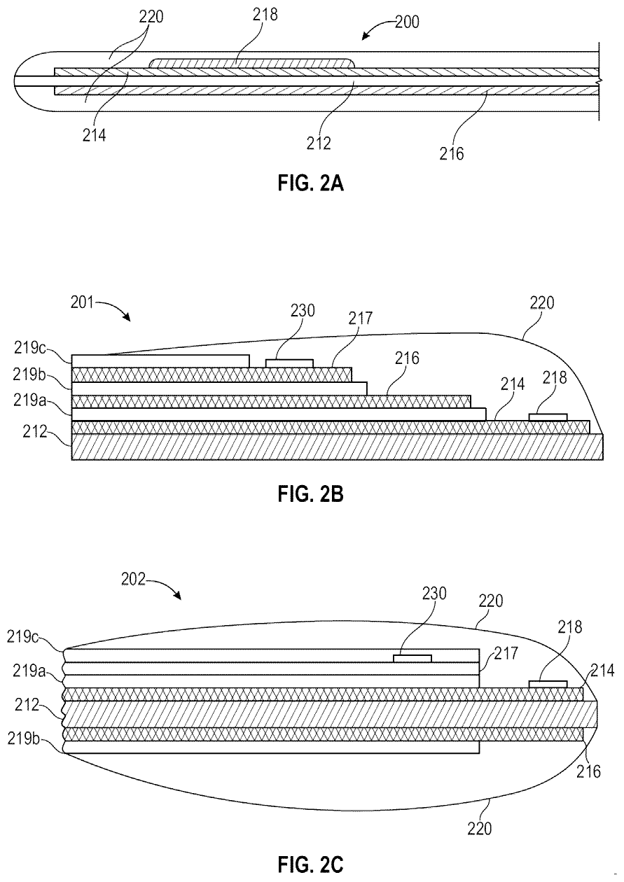

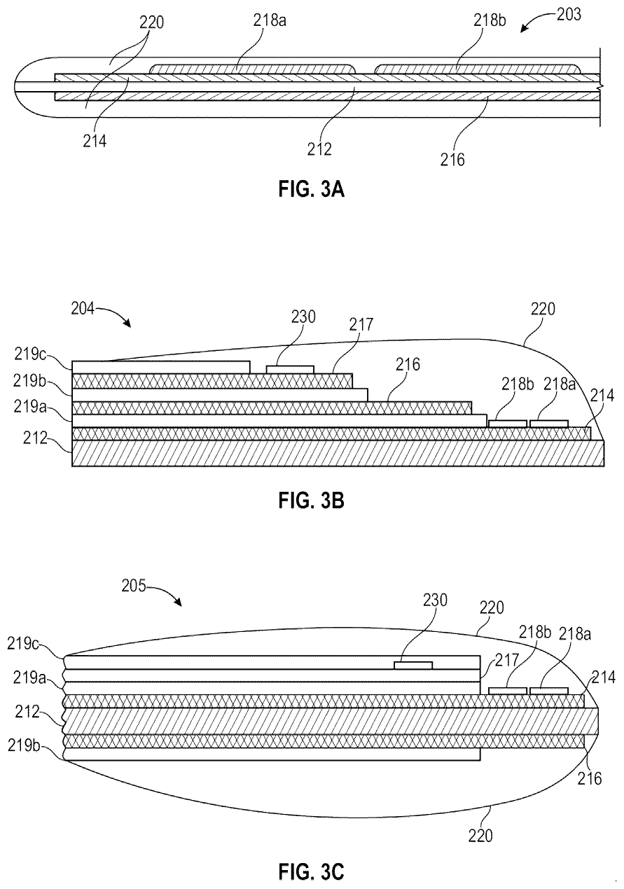

Sensor configurations featuring a single sensing layer that is configured for the detection of a corresponding single analyte can employ two-electrode or three-electrode detection motifs, as described further herein in reference to FIGS. 2A-2C. Sensor configurations featuring two different sensing layers for detection of separate analytes, either upon separate working electrodes or upon the same working electrode, are described separately thereafter in reference to FIGS. 3A-5C. Sensor configurations having multiple working electrodes can be particularly advantageous for incorporating two different sensing layers within the same sensor tail, since the signal contribution from each sensing layer can be determined more readily.

When a single working electrode is present in an analyte sensor, three-electrode sensor configurations can include a working electrode, a counter electrode, and a reference electrode. Related two-electrode sensor configurations can include a working electrode and a second electrode, in which the second electrode can function as both a counter electrode and a reference electrode (i.e., a counter/reference electrode). The various electrodes can be at least partially stacked (layered) upon one another and/or laterally spaced apart from one another upon the sensor tail. Suitable sensor configurations can be substantially flat in shape, substantially cylindrical in shape or any other suitable shape. In any of the sensor configurations disclosed herein, the various electrodes can be electrically isolated from one another by a dielectric material or similar insulator.

Analyte sensors featuring multiple working electrodes can similarly include at least one additional electrode. When one additional electrode is present, the one additional electrode can function as a counter/reference electrode for each of the multiple working electrodes. When two additional electrodes are present, one of the additional electrodes can function as a counter electrode for each of the multiple working electrodes and the other of the additional electrodes can function as a reference electrode for each of the multiple working electrodes.

FIG. 2A shows a diagram of an illustrative two-electrode analyte sensor configuration, which is compatible for use in the disclosure herein. As shown, analyte sensor 200 includes substrate 212 disposed between working electrode 214 and counter/reference electrode 216. Alternately, working electrode 214 and counter/reference electrode 216 can be located upon the same side of substrate 212 with a dielectric material interposed in between (configuration not shown). Sensing layer 218 is disposed as at least one layer upon at least a portion of working electrode 214. Sensing layer 218 can include multiple spots or a single spot configured for detection of an analyte (e.g., ketone), as discussed further herein.

Referring still to FIG. 2A, membrane 220 overcoats at least sensing layer 218. In certain aspects, membrane 220 can also overcoat some or all of working electrode 214 and/or counter/reference electrode 216, or the entirety of analyte sensor 200. One or both faces of analyte sensor 200 can be overcoated with membrane 220. Membrane 220 can include one or more polymeric membrane materials having capabilities of limiting analyte flux to sensing layer 218 (i.e., membrane 220 is a mass transport limiting membrane having some permeability for the analyte of interest). In some aspects, and further described below, membrane 220 is not crosslinked. Analyte sensor 200 can be operable for assaying an analyte (e.g., ketone) by any of coulometric, amperometric, voltammetric, or potentiometric electrochemical detection techniques.

FIGS. 2B and 2C show diagrams of illustrative three-electrode analyte sensor configurations, which are also compatible for use in the disclosure herein. Three-electrode analyte sensor configurations can be similar to that shown for analyte sensor 200 in FIG. 2A, except for the inclusion of additional electrode 217 in analyte sensors 201 and 202 (FIGS. 2B and 2C). With additional electrode 217, counter/reference electrode 216 can then function as either a counter electrode or a reference electrode, and additional electrode 217 fulfills the other electrode function not otherwise accounted for. Working electrode 214 continues to fulfill its original function. Additional electrode 217 can be disposed upon either working electrode 214 or electrode 216, with a separating layer of dielectric material in between. For example, and not by the way of limitation, as depicted in FIG. 2B, dielectric layers 219a, 219b and 219c separate electrodes 214, 216 and 217 from one another and provide electrical isolation. Alternatively, at least one of electrodes 214, 216 and 217 can be located upon opposite faces of substrate 212, as shown in FIG. 2C. Thus, in certain aspects, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material. Reference material layer 230 (e.g., Ag/AgCl) can be present upon electrode 217, with the location of reference material layer 230 not being limited to that depicted in FIGS. 2B and 2C. As with sensor 200 shown in FIG. 2A, sensing layer 218 in analyte sensors 201 and 202 can include multiple spots or a single spot. Additionally, analyte sensors 201 and 202 can be operable for assaying an analyte by any of coulometric, amperometric, voltammetric, or potentiometric electrochemical detection techniques.

Like analyte sensor 200, membrane 220 can also overcoat sensing layer 218, as well as other sensor components, in analyte sensors 201 and 202, thereby serving as a mass transport limiting membrane. In certain aspects, the additional electrode 217 can be overcoated with membrane 220. Although FIGS. 2B and 2C have depicted electrodes 214, 216, and 217 as being overcoated with membrane 220, it is to be recognized that in certain aspects only working electrode 214 is overcoated. Moreover, the thickness of membrane 220 at each of electrodes 214, 216, and 217 can be the same or different. As in two-electrode analyte sensor configurations (FIG. 2A), one or both faces of analyte sensors 201 and 202 can be overcoated with membrane 220 in the sensor configurations of FIGS. 2B and 2C, or the entirety of analyte sensors 201 and 202 can be overcoated. Accordingly, the three-electrode sensor configurations shown in FIGS. 2B and 2C should be understood as being non-limiting of the aspects disclosed herein, with alternative electrode and/or layer configurations remaining within the scope of the present disclosure.

FIG. 3A shows an illustrative configuration for sensor 203 having a single working electrode with two different sensing layers disposed thereon. FIG. 3A is similar to FIG. 2A, except for the presence of two sensing layers upon working electrode 214: first sensing layer 218a and second sensing layer 218b, which are responsive to different analytes and are laterally spaced apart from one another upon the surface of working electrode 214. Sensing layers 218a and 218b can include multiple spots or a single spot configured for detection of each analyte. The composition of membrane 220 can vary or be compositionally the same at sensing layers 218a and 218b. First sensing layer 218a and second sensing layer 218b can be configured to detect their corresponding analytes at working electrode potentials that differ from one another, as discussed further below.

FIGS. 3B and 3C show cross-sectional diagrams of illustrative three-electrode sensor configurations for sensors 204 and 205, respectively, each featuring a single working electrode having first sensing layer 218a and second sensing layer 218b disposed thereon. FIGS. 3B and 3C are otherwise similar to FIGS. 2B and 2C and can be better understood by reference thereto. As with FIG. 3A, the composition of membrane 220 can vary or be compositionally the same at sensing layers 218a and 218b.

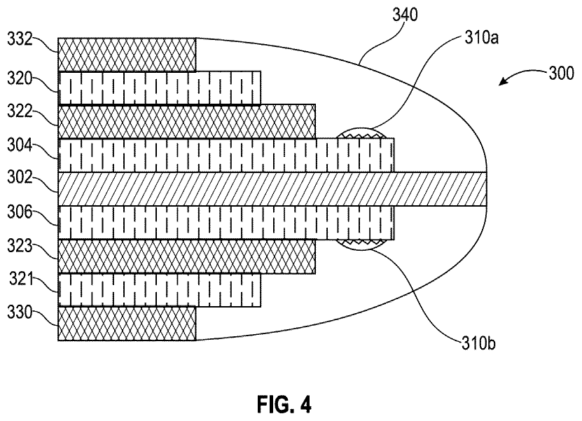

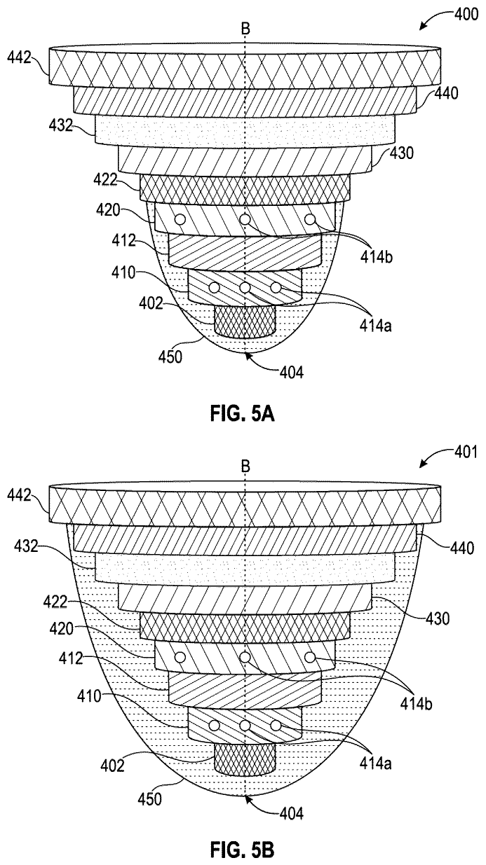

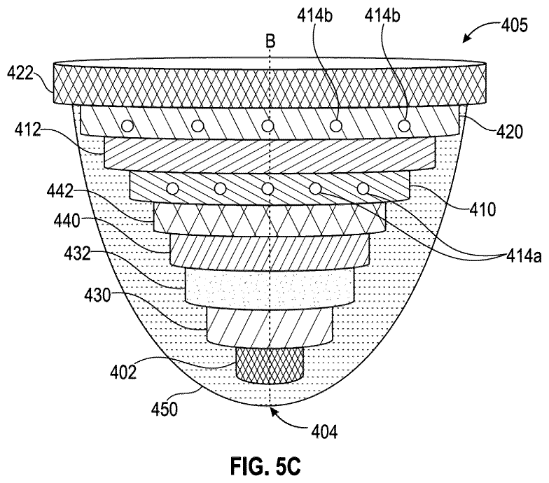

Illustrative sensor configurations having multiple working electrodes, specifically two working electrodes, are described in further detail in reference to FIGS. 4-5C. Although the following description is primarily directed to sensor configurations having two working electrodes, it is to be appreciated that more than two working electrodes can be incorporated through extension of the disclosure herein. Additional working electrodes can be used to impart additional sensing capabilities to the analyte sensors beyond just a first analyte and a second analyte, e.g., for the detection of a third and/or fourth analyte.

FIG. 4 shows a cross-sectional diagram of an illustrative analyte sensor configuration having two working electrodes, a reference electrode and a counter electrode, which is compatible for use in the disclosure herein. As shown, analyte sensor 300 includes working electrodes 304 and 306 disposed upon opposite faces of substrate 302. First sensing layer 310a is disposed upon the surface of working electrode 304, and second sensing layer 310b is disposed upon the surface of working electrode 306. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 can overcoat at least sensing layers 310a and 310b, according to various aspects, with other components of analyte sensor 300 or the entirety of analyte sensor 300 optionally being overcoated with membrane 340.

Like analyte sensors 200, 201, and 202, analyte sensor 300 can be operable for assaying an analyte (e.g., ketone) by any of coulometric, amperometric, voltammetric, or potentiometric electrochemical detection techniques.

Alternative sensor configurations having multiple working electrodes and differing from the configuration shown in FIG. 4 can feature a counter/reference electrode instead of separate counter and reference electrodes 320, 321, and/or feature layer and/or membrane arrangements varying from those expressly depicted. For example, and not by the way of limitation the positioning of counter electrode 320 and reference electrode 321 can be reversed from that depicted in FIG. 4. In addition, working electrodes 304 and 306 need not necessarily reside upon opposing faces of substrate 302 in the manner shown in FIG. 4.

Although suitable sensor configurations can feature electrodes that are substantially planar in character, it is to be appreciated that sensor configurations featuring non-planar electrodes can be advantageous and particularly suitable for use in the disclosure herein. In particular, substantially cylindrical electrodes that are disposed concentrically with respect to one another can facilitate deposition of a mass transport limiting membrane, as described hereinbelow. FIGS. 5A-5C show perspective views of analyte sensors featuring two working electrodes that are disposed concentrically with respect to one another. It is to be appreciated that sensor configurations having a concentric electrode disposition but lacking a second working electrode are also possible in the present disclosure.

FIG. 5A shows a perspective view of an illustrative sensor configuration in which multiple electrodes are substantially cylindrical and are disposed concentrically with respect to one another about a central substrate. As shown, analyte sensor 400 includes central substrate 402 about which all electrodes and dielectric layers are disposed concentrically with respect to one another. In particular, working electrode 410 is disposed upon the surface of central substrate 402, and dielectric layer 412 is disposed upon a portion of working electrode 410 distal to sensor tip 404. Working electrode 420 is disposed upon dielectric layer 412, and dielectric layer 422 is disposed upon a portion of working electrode 420 distal to sensor tip 404. Counter electrode 430 is disposed upon dielectric layer 422, and dielectric layer 432 is disposed upon a portion of counter electrode 430 distal to sensor tip 404. Reference electrode 440 is disposed upon dielectric layer 432, and dielectric layer 442 is disposed upon a portion of reference electrode 440 distal to sensor tip 404. As such, exposed surfaces of working electrode 410, working electrode 420, counter electrode 430, and reference electrode 440 are spaced apart from one another along longitudinal axis B of analyte sensor 400.

Referring still to FIG. 5A, first sensing layers 414a and second sensing layers 414b, which are responsive to different analytes or the same analyte, are disposed upon the exposed surfaces of working electrodes 410 and 420, respectively, thereby allowing contact with a fluid to take place for sensing. Although sensing layers 414a and 414b have been depicted as three discrete spots in FIG. 5A, it is to be appreciated that fewer or greater than three spots, including a continuous layer of sensing layer, can be present in alternative sensor configurations.

In FIG. 5A, sensor 400 is partially coated with membrane 450 upon working electrodes 410 and 420 and sensing layers 414a and 414b disposed thereon. FIG. 5B shows an alternative sensor configuration in which the substantial entirety of sensor 401 is overcoated with membrane 450. Membrane 450 can be the same or vary compositionally at sensing layers 414a and 414b.

It is to be further appreciated that the positioning of the various electrodes in FIGS. 5A and 5B can differ from that expressly depicted. For example, the positions of counter electrode 430 and reference electrode 440 can be reversed from the depicted configurations in FIGS. 5A and 5B. Similarly, the positions of working electrodes 410 and 420 are not limited to those that are expressly depicted in FIGS. 5A and 5B. FIG. 5C shows an alternative sensor configuration to that shown in FIG. 5B, in which sensor 405 contains counter electrode 430 and reference electrode 440 that are located more proximal to sensor tip 404 and working electrodes 410 and 420 that are located more distal to sensor tip 404. Sensor configurations in which working electrodes 410 and 420 are located more distal to sensor tip 404 can be advantageous by providing a larger surface area for deposition of sensing layers 414a and 414b (five discrete sensing spots illustratively shown in FIG. 5C), thereby facilitating an increased signal strength in some cases. Similarly, central substrate 402 can be omitted in any concentric sensor configuration disclosed herein, wherein the innermost electrode can instead support subsequently deposited layers.

Several parts of the sensor are further described below.

The present disclosure relates to a ketone sensor and methods of detecting ketone with high sensitivity so that lower concentrations of ketone can be measured. It was surprisingly discovered that sensitivity can be improved by lowering the applied potential, subtracting background interference, and by accumulating charge.

Without wishing to be bound by theory, because cumulative detection involves alternating connection and disconnection of the sensing electrode, in some aspects, it can be possible to use a single channel that switches between background and ketone sensing chemistry (e.g. switching between WE1 (standard chemistry) and WE2 (blank chemistry) about every 2.5 minutes). Thus, the redox mediator can act as a “data storage medium” as it builds up charge, resulting in a simplified design of the electronics by requiring only one channel for background storage rather than two channels. This set up can introduce a time offset between the background (blank) and ketone (standard) measurements that could complicate background subtraction, but this offset can be short enough to prevent significant changes in analyte and interference concentrations between measurements. Alternatively, the single channel can be connected to the first working electrode (WE1) for a short amount of time (e.g., on the order of seconds, such as about 15 seconds), connected to the second working electrode (WE2) for a short amount of time (e.g., on the order of seconds, such as about 15 seconds), and then left disconnected from both channels for a longer amount of time (e.g., on the order of minutes, such as about 2 minutes) to provide similar peak currents to the previous example but reducing the time offset between channels from minutes (e.g., about 2.5 minutes) to seconds (e.g., about 15 seconds).

In some aspects of the ketone sensing method, a ketone-sensing electrode and background sensing electrode are both connected to a circuit and a potential is applied. The period of time both sensing electrodes are connected to the circuit can be any suitable time period that allows for detecting a ketone and/or background signal. Typically, the period of time for both sensing electrodes to be connected (e.g., a connecting set period of time) can be about 1 second or more (e.g., about 2 seconds or more, about 3 seconds or more, about 4 seconds or more, about 5 seconds or more, about 10 seconds or more, about 20 seconds or more, about 30 seconds or more, about 40 seconds or more, about 50 seconds or more, about 1 minute or more, about 2 minutes or more, about 3 minutes or more, about 4 minutes or more, about 5 minutes or more, about 6 minutes or more, about 7 minutes or more, about 8 minutes or more, or about 10 minutes or more) to about 30 minutes or less (e.g., about 25 minutes or less, about 20 minutes or less, about 15 minutes or less, about 10 minutes or less, about 8 minutes or less, about 7 minutes or less, about 6 minutes or less, about 5 minutes or less, about 4 minutes or less, about 3 minutes or less, about 2 minutes or less, about 1 minute or less, about 50 seconds or less, about 40 seconds or less, about 30 seconds or less, about 20 seconds or less, about 10 seconds or less, or about 5 seconds or less). In some aspects, the connecting period of time is about 1 second to about 2 minutes or about 5 seconds to about 1 minute. Both electrodes are subsequently disconnected from the circuit to allow the charge to accumulate. After sufficient charge has been built up, both electrodes can be reconnected to the circuit, where a ketone signal (i.e., a measure of ketone signal+background signal) and a background signal (i.e., a measure of background signal only) are both measured. The ketone concentration can be correlated to the measured ketone signal minus the measured background signal.

In one aspect, the present disclosure is directed to a method for sensing ketone comprising:
contacting a biofluid comprising a ketone with a first sensing electrode comprising a ketone-responsive enzyme and a redox mediator and a second sensing electrode comprising a redox mediator and no ketone-responsive enzyme;
connecting the first and second sensing electrodes to a circuit and applying a potential less than +40 mV to both electrodes to provide a steady state;
disconnecting the first and second sensing electrodes from the circuit;
accumulating a charge derived from the biofluid reacting with the first and second sensing electrodes for a set period of time (e.g., an accumulating set period of time);
connecting (e.g., reconnecting) the first and second sensing electrodes to the circuit after the set period of time; and
measuring a ketone signal by subtracting a signal obtained from the second sensing electrode from a signal obtained from the first sensing electrode.

In some aspects of the ketone sensing method, the connecting step and disconnecting/accumulating step alternate between a ketone-sensing electrode and a background sensing electrode. In particular, the present disclosure is directed to a method for sensing ketone comprising:
(a) contacting first and second sensing electrodes with a biofluid comprising a ketone, wherein the first sensing electrode comprises a ketone-responsive enzyme and a redox mediator and the second sensing electrode comprises a redox mediator and no ketone-responsive enzyme;
(b) connecting the first sensing electrode to a circuit and applying a potential less than +40 mV to provide a steady state;
(c) disconnecting the first sensing electrode from the circuit and connecting the second sensing electrode to the circuit and applying a potential less than +40 mV to provide a steady state;
(d) accumulating a charge derived from the biofluid reacting with the first sensing electrode for a first set period of time;
(e) connecting (e.g., reconnecting) the first sensing electrode to the circuit after the first set period of time and disconnecting the second sensing electrode from the circuit;
(f) accumulating a charge derived from the biofluid reacting with the second sensing electrode for a second set period of time;
(g) connecting (e.g., reconnecting) the second sensing electrode to the circuit after the second set period of time; and
(h) measuring a ketone signal by subtracting a signal obtained from the second sensing electrode from a signal obtained from the first sensing electrode.

In some aspects of the ketone sensing method, a ketone-sensing electrode and background sensing electrode are both contacted with a biofluid that comprises a ketone. The period of time both sensing electrodes are contacted with the fluid can be any suitable time period that allows for detecting a ketone and/or background signal. Typically, the period of time for both sensing electrodes to be contacted to a biofluid (e.g., a contacting set period of time) can be about 1 second or more (e.g., about 2 seconds or more, about 3 seconds or more, about 4 seconds or more, about 5 seconds or more, about 10 seconds or more, about 20 seconds or more, about 30 seconds or more, about 40 seconds or more, about 50 seconds or more, about 1 minute or more, about 2 minutes or more, about 3 minutes or more, about 4 minutes or more, about 5 minutes or more, about 6 minutes or more, about 7 minutes or more, about 8 minutes or more, or about 10 minutes or more) to about 30 minutes or less (e.g., about 25 minutes or less, about 20 minutes or less, about 15 minutes or less, about 10 minutes or less, about 8 minutes or less, about 7 minutes or less, about 6 minutes or less, about 5 minutes or less, about 4 minutes or less, about 3 minutes or less, about 2 minutes or less, about 1 minute or less, about 50 seconds or less, about 40 seconds or less, about 30 seconds or less, about 20 seconds or less, about 10 seconds or less, or about 5 seconds or less). In some aspects, the contacting period of time is about 1 second to about 2 minutes or about 5 seconds to about 1 minute.

In some aspects, the measuring step can comprise determining the concentration of ketone in the fluid (e.g., a biofluid), which can be correlated by subtracting the signal obtained from the second sensing electrode (second signal) from the signal obtained from the first sensing electrode (first signal). In some aspects, ketone concentration (mM) can be determined by the formula:

(ketone signal (nA)−blank signal (nA))/ketone response (nA/mM).

Known ketone concentrations can be added to a control sample to determine a ketone response value (nA/mM) for a sensor.

The disconnecting and connecting of step (b) and step (c) can be simultaneous or sequential. In some aspects of this alternating method, the disconnecting and connecting of steps (b) and (c) are simultaneous.

The accumulating set period of time, which includes the first set period of time and the second set period of time, can be any suitable time period that allows for accumulating adequate charge to detect ketone and/or the background once the circuit is reconnected. Typically, the set period of time can be about 30 seconds or more (e.g., 1 minute or more, 2 minutes or more, 3 minutes or more, 4 minutes or more, 5 minutes or more, 6 minutes or more, 7 minutes or more, 8 minutes or more, 10 minutes or more, 15 minutes or more, 20 minutes or more, 25 minutes or more). In general, the set period of time can be about 30 minutes or less (e.g., 25 minutes or less, 20 minutes or less, 15 minutes or less, 10 minutes or less, 8 minutes or less, 7 minutes or less, 6 minutes or less, 5 minutes or less, 4 minutes or less, 3 minutes or less, 2 minutes or less, or 1 minute or less) to allow for complete reaction of all of the ketone present at the ketone sensing electrode, the reaction of interferents at the background sensing electrode, or both. For example, the set period of time can be about 30 seconds or more and about 30 minutes or less, about 1 to about 20 minutes, about 1 to about 15 minutes, about 1 to about 10 minutes, about 1 to about 8 minutes, about 1 to about 5 minutes, about 2 to about 8 minutes, about 2 to about 5 minutes, or about 30 seconds or more. The first set period of time can be the same or different from the second set period of time. In some aspects, the first set period of time can be the same as the second set period of time. In other aspects, the first set period of time can be different from the second set period of time. In any of these aspects, the set period of time, the first set period of time, the second set period of time, or any combination thereof can be 30 seconds or more.

It was surprisingly discovered that lowering the sensing potential reduced the interaction between the sensing electrode and background interferents (e.g., electroactive interferents), which in turn decreased interference signal. In some aspects, the potential applied to the ketone sensor can be less than +40 mV to about −250 mV, including ranges of less than +40 mV to about −225 mV, less than +40 mV to about −200 mV, less than +40 mV to about −175 mV, less than +40 mV to about −150 mV, less than +40 mV to about −125 mV, about +30 mV to about −250 mV, about +30 mV to about −225 mV, about +30 mV to about −200 mV, about +30 mV to about −175 mV, about +30 mV to about −150 mV, about +30 mV to about −125 mV, about +20 mV to about −250 mV, about +20 mV to about −225 mV, about +20 mV to about −200 mV, about +20 mV to about −175 mV, about +20 mV to about −150 mV, about +20 mV to about −125 mV, about +10 mV to about −250 mV, about +10 mV to about −225 mV, about +10 mV to about −200 mV, about +10 mV to about −175 mV, about +10 mV to about −150 mV, about +10 mV to about −125 mV, about +5 mV to about −250 mV, about +5 mV to about −225 mV, about +5 mV to about −200 mV, about +5 mV to about −175 mV, about +5 mV to about −150 mV, about +5 mV to about −125 mV, each relative to an Ag/AgCl reference. In some aspects, the applied potential can be about +5 mV to about −125 mV, about −5 mV to about −100 mV, about −10 mV to about −90 mV, or about −20 mV to about −80 mV, each relative to an Ag/AgCl reference. In some aspects, the potential applied can be about +35 mV, about +30 mV, about +25 mV, about +20 mV, about +15 mV, about +10 mV, about +5 mV, about −5 mV, about −10 mV, about −15 mV, about −20 mV, about −25 mV, about −30 mV, about −40 mV, about −50 mV, about −60 mV, about −70 mV, about −80 mV, about −90 mV, about −100 mV, about −110 mV, about −120 mV, about −130 mV, about −140 mV, about −150 mV, about −160 mV, about −170 mV, about −180 mV, about −190 mV, about −200 mV, about −210 mV, about −220 mV, about −230 mV, about −240 mV, or about −250 mV, each relative to an Ag/AgCl reference. In some particular aspects, the potential applied can be about −80 mV vs Ag/AgCl.

In any of these aspects, the sensing electrodes can comprise a working electrode and either a ketone sensing layer or a background sensing layer on a portion of a working electrode. In the ketone sensor, a working electrode (e.g., the working electrode in the first sensing electrode, the working electrode in the second sensing electrode) can be any suitable conductive material. Examples of suitable conductive materials include, e.g., aluminum, carbon (including graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements. In some aspects, a working electrode (e.g., the working electrode in the first sensing electrode, the working electrode in the second sensing electrode) can comprise carbon.

In the first sensing electrode, the ketone sensing layer disposed on at least a portion of the working electrode senses ketone and comprises a ketone-responsive enzyme and a redox mediator. In some aspects, the ketone that will be sensed can be an endogenous ketone. For example, the ketone to be sensed can be acetone, acetoacetic acid, acetoacetate, β-hydroxybutyric acid (β-HBA), or any combination thereof. β-Hydroxybutyric acid (β-HBA) is technically a carboxylic acid rather than a ketone but is commonly referred to in the field as a blood ketone. In some aspects, the ketone to be sensed can be acetoacetate, β-HBA, β-hydroxybutyrate (β-HB), or any combination thereof. In some aspects, the ketone to be sensed can be β-hydroxybutyrate (β-HB).

The ketone sensing layer comprises a ketone-responsive enzyme, which serves as a catalyst for the electron transfer. In some aspects, the ketone-responsive enzyme can be 3-hydroxybutyrate dehydrogenase (3-HBDH), glucose dehydrogenase, an alcohol dehydrogenase, or combinations thereof. In some aspects, the ketone-responsive enzyme can be 3-hydroxybutyrate dehydrogenase (3-HBDH).

In some aspects, the first sensing electrode can comprise an NAD(P)H oxidoreductase, such as diaphorase. If necessary, one or more cofactors can be included with the ketone-responsive enzyme or NAD(P)H oxidoreductase enzyme. Suitable cofactors include, e.g., nicotinamide adenine dinucleotide, in either oxidized (NAD) or reduced form (NADH), or a derivative thereof. Without wishing to be bound by theory, it is believed that a ketone-responsive enzyme, such as 3-HBDH, cannot easily directly pass electrons to the redox mediator. Thus, an NAD(P)H oxidoreductase (e.g., diaphorase) and an optional cofactor (e.g., NAD) can be added to shuttle electrons from the ketone-responsive enzyme to the redox mediator. In any of these aspects, the first sensing electrode can comprise a ketone-responsive enzyme (e.g., 3-HBDH), an NAD(P)H oxidoreductase, and nicotinamide adenine dinucleotide phosphate (NAD(P)+) or a derivative thereof.

In some aspects, the ketone sensing layer can further comprise an albumin, which can act as an enzyme stabilizer. In an aspect, the albumin can be a serum albumin, such as bovine serum albumin (BSA) or human serum albumin (HSA). In certain aspects, the sensing layer can comprise human serum albumin.

In certain aspects, the sensing layer can include a ratio of albumin stabilizer to enzyme (e.g., 3-HBDH) from about 40:1 to about 1:40, e.g., from about 35:1 to about 1:35, from about 30:1 to about 1:30, from about 25:1 to about 1:25, from about 20:1 to about 1:20, from about 15:1 to about 1:15, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2 or about 1:1. In certain aspects, the sensing layer can include a ratio of albumin stabilizer to enzyme from about 1:1 to about 1:10, e.g., from about 1:1 to about 1:9, from about 1:1 to about 1:8, from about 1:1 to about 1:7, from about 1:1 to about 1:6, from about 1:1 to about 1:5, from about 1:2 to about 1:9, from about 1:3 to about 1:8, from about 1:3 to about 1:7 or from about 1:4 to about 1:6.

In a specific example, β-hydroxybutyrate dehydrogenase can convert β-hydroxybutyrate and oxidized nicotinamide adenine dinucleotide (NAD+) into acetoacetate and reduced nicotinamide adenine dinucleotide (NADH), respectively. The enzyme cofactors NAD and NADH can aid in promoting the concerted enzymatic reactions disclosed herein. The NADH can then undergo reduction under diaphorase mediation, with the electrons transferred during this process providing the basis for ketone detection at the working electrode. The electrochemical signal obtained can then be correlated to the amount of ketone that was initially present in the sample at the time of measurement. Thus, there is a 1:1 molar correspondence between the amount of electrons transferred to the working electrode and the amount of β-hydroxybutyrate converted, thereby providing the basis for ketones detection and quantification based upon the measured amount of current at the working electrode. Transfer of the electrons resulting from NADH reduction to the working electrode can take place through an electron transfer agent, such as an osmium (Os) compound, as described herein. An albumin can optionally be present as a stabilizer for the enzymes.

The ketone-responsive enzyme can be present in any suitable amount, including from about 1% to about 50% by weight (e.g., about 1% to about 40% by weight, about 1% to about 30% by weight, about 1% to about 20% by weight, about 1% to about 15% by weight, about 1% to about 10% by weight, or from about 1% to about 5% by weight) relative to the redox mediator.

The NAD(P)H oxidoreductase (e.g., diaphorase) can be present in the ketone sensing layer in any suitable amount, including from about 0.01% to 10% by weight (e.g., about 0.05% to about 9.5% by weight, about 0.1% to about 9% by weight, about 0.5% to about 8.5% by weight, about 1% to about 8% by weight, or about 2% to about 7% by weight) of the total enzyme composition.

In any of the aspects, the ketone sensing layer can comprise a pH buffer. The buffer can be any suitable composition that is water soluble and controls (i.e., maintains) the pH of the sensing composition within a pH of about 5 to about 8 (e.g., maintains a pH of about 5, about 5.5, about 6, about 6.5, about 7, about 7.5, or about 8). In some aspects, the pH can be controlled to be within a range of about 6 to about 8. For example, the buffer can comprise a phosphate (e.g., monobasic and dibasic sodium phosphate), 4-(2-hydroxyethyl) piperazine-1-ethanesulfonic acid (HEPES), 2-(N-morpholino) ethanesulfonic acid (MES), 3-(N-morpholino) propanesulfonic acid (MOPS), 2-amino-2-(hydroxymethyl)-1,3-propanediol (TRIS), a carbonate (e.g., carbonic acid and a carbonate salt, such as sodium carbonate; sodium carbonate and sodium bicarbonate), or a citrate (e.g., citric acid and a citrate salt, such as trisodium citrate). The buffer can optionally comprise one or more (e.g., 1, 2, 3, or 4) additional salts (e.g., Group I or Group II halide salts, e.g., sodium chloride, potassium chloride, magnesium chloride). In an aspect, the buffer can be phosphate-buffered saline (PBS), which comprises disodium hydrogen phosphate, sodium chloride, and optionally potassium chloride and potassium dihydrogen phosphate. In another aspect, the buffer can be MES or a phosphate buffer, which can comprise phosphate, sodium chloride, potassium chloride, and/or magnesium chloride.

In some aspects, the buffer typically can be an aqueous buffer. In other aspects, non-aqueous solvents can be present, such as an alcohol (e.g., ethanol). In some aspects, the buffer can comprise water as the only solvent. In other aspects, the buffer can comprise water and at least one (e.g., 1, 2, or 3) non-aqueous solvents in any suitable ratio, such as a non-aqueous solvent to water volume ratio ranging from 99.9:0.1 to 0.1:99.9. In some aspects, the non-aqueous solvent to water volume ratio can be about 1:99, about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, or about 99:1, etc.). In a specific example, ethanol (EtOH) and water can be used in a volume ratio ranging from 50:50 to 90:10 EtOH:H₂O (e.g., 70:30, about 75:25, about 80:20, about 85:15, or about 90:10, etc.).

In aspects, the second sensing electrode is a background sensing electrode, which comprises a working electrode and a background sensing layer on a portion of the working electrode. As described herein, the background sensing layer sense the background interference and comprises a redox mediator but no ketone-responsive enzyme. The redox material in the first and second sensing electrodes can be the same or different. In an aspect, the redox mediator in the first and second sensing electrodes are the same material. In an aspect, the background sensing layer does not comprise 3-HBDH. Optional components, such as NAD(P)H oxidoreductase (e.g., diaphorase), one or more cofactors, an albumin (e.g., human serum albumin (HSA)), a pH buffer, each as described herein, can be present in the background sensing layer. In some aspects, the background sensing layer can comprise an albumin (e.g., HSA), an NAD(P)H oxidoreductase (e.g., diaphorase) and nicotinamide adenine dinucleotide, in either oxidized (NAD) or reduced form (NADH), or a derivative thereof. In some aspects, the background sensing layer can comprise human serum albumin, diaphorase and nicotinamide adenine dinucleotide in oxidized form (NAD).

In some aspects, the background sensing layer comprises a ketone-responsive enzyme (e.g., the ketone-responsive enzyme is the only enzyme) but does not comprise any other sensing components, such as an albumin (e.g., HSA), an NAD(P)H oxidoreductase (e.g., diaphorase), a nicotinamide adenine dinucleotide, in either oxidized (NAD) or reduced form (NADH), or a derivative thereof, or a redox mediator. In some aspects, the background sensing layer consists of a ketone-responsive enzyme.

In some aspects, the background sensing layer does not comprise an albumin (e.g., HSA). In some aspects, the background sensing layer does not comprise diaphorase. In some aspects, the background sensing layer does not comprise added NAD or NADH. In some aspects, the background sensing layer comprises ambient NAD and NADH in the complete or substantial absence of added NAD and/or NADH. In some aspects, the background sensing layer does not comprise a redox mediator.

In some aspects, the background sensing layer does not comprise a ketone-responsive enzyme or an albumin (e.g., HSA). In some aspects, the background sensing layer does not comprise a ketone-responsive enzyme or diaphorase. In some aspects, the background sensing layer does not comprise a ketone-responsive enzyme or added NAD or NADH. In some aspects, the background sensing layer does not comprise a ketone-responsive enzyme or a redox mediator.

In some aspects, the background sensing layer does not comprise a ketone-responsive enzyme, an albumin (e.g., HSA), or diaphorase. In some aspects, the background sensing layer does not comprise a ketone-responsive enzyme, diaphorase, or added NAD. In some aspects, the background sensing layer does not comprise a ketone-responsive enzyme, an albumin (e.g., HSA), or diaphorase. In some aspects, the background sensing layer does not comprise a ketone-responsive enzyme, diaphorase, or added NAD.

The ketone sensing layer and/or the background sensing layer can be continuously or discontinuously disposed on at least a portion of the respective working electrode. A discontinuous application means that the sensing layer can form a discrete shape on the working electrode, such as a spot, a line, or a plurality (e.g., an array) of spots and/or lines. The number of spots or lines is not considered to be particularly limited, but can range from 2 to about 10 (e.g., 2, 3, 4, 5, 6, 7, 8, 9, or 10, including about 3 to about 8, or from about 4 to about 6). In any of the aspects herein, the ketone sensing layer, the background sensing layer, or both can be continuous on the respective working electrode. In other aspects, the sensing layer, the background sensing layer, or both can be discontinuous on the respective working electrode.

The total size of the creatinine sensing layer or layers (e.g., combined area of all spots, layers, or active areas) can be at least about 0.05 mm² and can be up to about 100 mm². In some aspects, the total size can be about 0.05 mm² to about 100 mm², about 0.05 mm² to about 75 mm², about 0.05 mm² to about 50 mm², about 0.05 mm² to about 40 mm², about 0.05 mm² to about 30 mm², about 0.05 mm² to about 25 mm², about 0.05 mm² to about 15 mm², about 0.05 mm² to about 10 mm², about 0.05 mm² to about 5 mm², about 0.05 mm² to about 1 mm², or about 0.05 mm² to about 0.1 mm². In a particular aspect, the total size of the sensing layer or layers ranges from about 0.05 to about 0.1 mm², about 0.05 to about 100 mm², about 0.1 to about 50 mm², about 0.5 to about 30 mm², about 1 to about 20 mm², or about 1 to about 15 mm².

Each sensing layer or layers, including the ketone sensing layer, the background sensing layer, or both, typically has a thickness that ranges from about 0.1-10 μm. For example, each sensing layer can be 0.1 μm thick or more (e.g., 0.2 μm or more, 0.3 μm or more, 0.5 μm or more, 0.8 μm or more, 1 μm or more, 2 μm or more, 3 μm or more, 5 μm or more, or 8 μm or more) and typically will have a thickness of 10 μm or less (e.g., 8 μm or less, 5 μm or less, 3 μm or less, 2 μm or less, 1 μm or less, 0.8 μm or less, 0.5 μm or less, 0.3 μm or less, or 0.2 μm or less). In an example, each layer present can have a thickness of about 0.1 to about 10 μm, about 0.2 to about 8 μm, about 0.5 to about 5 μm, about 1 to about 4 μm, or about 2 μm.

In some aspects, a conductive material such as, for example, carbon nanotubes, graphene, or metal nanoparticles, can be combined within the sensing layer or layers, including the ketone sensing layer, the background sensing layer, or both, to promote rapid attainment of a steady state current. Conductive material can be included in a range from about 0.1% to about 50% by weight (pbw) of each sensing layer (e.g., about 1 to about 50 pbw, about 1 to about 10 pbw, or about 0.1 to about 10 pbw).

Both the first sensing electrode (e.g., ketone sensing layer) and the second sensing electrode (e.g., background sensing layer) comprise a redox mediator. Each redox mediator can be the same or different. In some aspects, the redox mediator can be the same for both the first and second sensing electrodes.

In an aspect, the redox mediator can comprise a polymer and an electron transfer agent.

In some aspects, the polymer in the redox mediator can be any suitable polymer that allows the transfer of electrons between the electron transfer agent and the working electrode. For example, the polymer can be a polyvinylpyridine (e.g., poly(4-vinylpyridine; PVP)), a polyvinylimidazole (e.g., poly(1-vinylimidazole; PVI)), poly(aniline), poly(pyrrole), poly(acetylene), poly(acrylic acid), styrene/maleic anhydride copolymer, methylvinylether/maleic anhydride copolymer, poly(vinylbenzylchloride), poly(allylamine), poly(lysine), poly(acrylamide-co-1-vinyl imidazole), poly(4-vinylpyridine) quaternized with carboxypentyl groups, or poly(sodium 4-styrene sulfonate). These polymers can be considered precursor polymers in that the polymers are further modified to immobilize (e.g., attach) the electron transfer complex. In some aspects, the polymer can comprise a backbone comprising poly(4-vinylpyridine), poly(l-vinylimidazole), poly(styrene), poly(thiophene), poly(aniline), poly(pyrrole), poly(acetylene), or any combination thereof. In other aspects, the polymer can comprise a polymer or copolymer repeat unit that can comprise at least one (e.g., 1, 2, 3, 4, 5, or 6) pendant pyridinyl group, imidazolyl group, or both a pyridinyl and imidazolyl group. For example, a suitable polymer can include partially or fully quaternized poly(4-vinylpyridine) and poly(1-vinylimidazole), in which quaternized pyridine and imidazole groups, respectively, can be used to form spacers by reaction with (e.g., complexation with) an electron transfer agent.

In some aspects, the electron transfer agent in the redox mediator can comprise a transition metal complex. The transition metal in the transition metal complex can be any suitable transition metal that can be effectively reduced and oxidized in the method described herein. For example, the transition metal complex can comprise osmium, ruthenium, iron, cobalt, vanadium, or a combination thereof. In some aspects, the transition metal can be ruthenium or osmium, particularly osmium. According to some aspects, suitable electron transfer agents can include low-potential osmium complexes, such as those described in U.S. Pat. Nos. 6,134,461, 6,605,200, 6,736,957, 7,501,053, and 7,754,093, the disclosures of each of which are incorporated herein by reference in their entirety. Other suitable examples of electron transfer mediators and polymer-bound electron transfer mediators can include those described in U.S. Pat. Nos. 8,444,834, 8,268,143, and 6,605,201, the disclosures of which are incorporated herein by reference in their entirety.

The transition metal complex can further comprise at least one ligand, which can be monodentate or multidentate (e.g., bidentate, tridentate, tetradentate). Typically, the complex will include enough ligands to provide a full coordination sphere. In some aspects, at least one ligand (e.g., 1, 2, 3, 4, 5, or 6) can comprise a nitrogen-containing heterocycle.

Monodentate ligands include, for example, —F, —Cl, —Br, —I, —CN, —SCN, —OH, NH₃, alkylamine, dialkylamine, trialkylamine, alkoxy, a heterocyclic compound, compounds containing such groups, a solvent molecule (e.g., H₂O, EtOH), or a reactive group. For example, an alkyl (e.g., C₁₋₁₂, C₁₋₆, C₁₋₄, C₁₋₃) or aryl (e.g., phenyl, benzyl, naphthyl) portions of a ligand can be optionally substituted by F, Cl, Br, I, alkylamino, dialkylamino, trialkylammonium (except aryl portions), alkoxy, alkylthio, aryl. Examples of suitable heterocyclic monodentate ligands include imidazole, pyrazole, oxazole, thiazole, pyridine, and pyrazine, each of which can be unsubstituted or substituted, as described herein (e.g., with at least one reactive group, such as 1, 2, 3, or 4 reactive groups).

Examples of suitable bidentate ligands include, for example, 1,10-phenanthroline, an amino acid, oxalic acid, acetylacetone, a diaminoalkane, an ortho-diaminoarene, 2,2′-biimidazole, 2,2′-bioxazole, 2,2′-bithiazole, 2-(2-pyridyl) imidazole, and 2,2′-bipyridine, each of which can be unsubstituted or substituted, as described herein (e.g., substituted with at least one reactive group, such as 1, 2, 3, or 4 reactive groups). Particularly suitable bidentate ligands for the electron transfer complex include substituted and unsubstituted 2,2′-biimidazole, 2-(2-pyridyl) imidazole, and 2,2′-bipyridine. Examples of suitable terdentate ligands include, for example, diethylenetriamine, 2,2′,2″-terpyridine, 2,6-bis(N-pyrazolyl)pyridine, each of which can substituted or unsubstituted (e.g., substituted with one more alkyl groups, such as methyl, or one or more reactive groups).

A suitable 2,2′-biimidazole ligand can be a ligand according to formula (I):

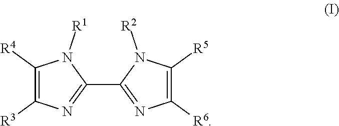

In formula (I), R¹ and R² are the same or different and each is a substituted or unsubstituted alkyl, alkenyl, or aryl. Generally, R¹ and R² are the same or different and each is an unsubstituted C₁₋₁₂ alkyl (e.g., C₁₋₄ alkyl). In some aspects, both R¹ and R² are methyl.

In formula (I), R³, R⁴, R⁵, and R⁶ are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Alternatively, R³ and R⁴, in combination, or R⁵ and R⁶, in combination, independently form a saturated or unsaturated 5- or 6-membered ring (e.g., benzo). Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H). Generally, R³, R⁴, R⁵, and R⁶ are the same or different and each is H or an unsubstituted C₁₋₁₂ alkyl (e.g., C₁₋₄ alkyl). In some aspects, R³, R⁴, R⁵, and R⁶ are all H.

A suitable 2-(2-pyridyl) imidazole ligand can be a ligand according to formula (II):

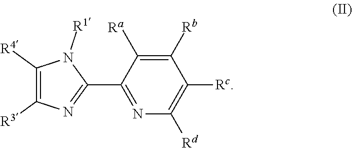

In formula (II), R¹ is a substituted or unsubstituted alkyl, alkenyl, or aryl. Generally, R¹ is an unsubstituted C₁₋₁₂ alkyl (e.g., C₁₋₄ alkyl) or a C₁₋₁₂ alkyl that is optionally substituted with a reactive group. In some aspects, R¹ is methyl.

In formula (II), R^{3′}, R^{4′}, R^{a}, R^{b}, R^{c}, and R^{d} are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Alternatively, R^{3′} and R^{4′}, in combination, or two adjacent substituents of R^{a}, R^{b}, R^{c}, and R^{d} (e.g., R^{a} and R^{b}, R^{b} and R^{c}, or R^{c} and R^{d}) in combination, independently form a saturated or unsaturated 5- or 6-membered ring (e.g., benzo). Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H). Generally, R^{3′}, R^{4′}, R^{a}, R^{b}, R^{c}, and R^{d} are the same or different and each is H or an unsubstituted C₁₋₁₂ alkyl (e.g., C₁₋₄ alkyl). In some aspects, R^{3′}, R⁴, R^{a}, R^{b}, Re, and R^{d} are all H.

A suitable 2,2′-bipyridine ligand can be a ligand according to formula (III):

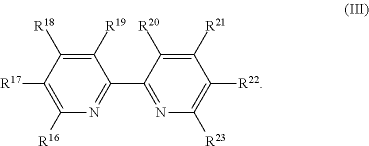

In formula (III), R¹⁶, R¹⁷, R¹⁸, R¹⁹, R²⁰, R²¹, R²², and R²³ are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H).

Specific examples of suitable combinations include R¹⁶ and R²³ are both H or both methyl and/or R¹⁷ and R²³ are both H or both methyl and/or R¹⁸ and R²¹ are both H or both methyl and/or R¹⁹ and R²⁰ are both H or both methyl. An alternative combination is where one or more adjacent pairs of substituents (e.g., R¹⁶ and R¹⁷, R¹⁷ and R¹⁸, R¹⁸ and R¹⁹, R²³ and R²², R²² and R²¹, or R²¹ and R²⁰), in combination, form a saturated or unsaturated 5- or 6-membered ring (e.g., benzo).

In an aspect, the one or more ligand is 4,4′-dimethyl-2,2′-bipyridine, mono-, di-, or polyalkoxy-2,2′-bipyridines (e.g., 4,4′-dimethoxy-2,2′-bipyridine), 4,7-dimethyl-1,10-phenanthroline, mono, di-, or polyalkoxy-1,10-phenanthrolines (e.g., 4,7-dimethoxy-1,10-phenanthroline), or a combination of any of these.

In some aspects, the transition metal complex can include a counterion (X) to balance the charge of the transition metal. Typically, there can be 1 to 5 (i.e., 1, 2, 3, 4, or 5) counterions. Multiple counterions in the complex are not necessarily all the same. Examples of suitable counterions include anions, such as halide (e.g., fluoride, chloride, bromide, or iodide), sulfate, phosphate, hexafluorophosphate, and tetrafluoroborate, and cations (e.g., a monovalent cation), such as lithium, sodium, potassium, tetralkylammonium, and ammonium. In some aspects, the counterion is a halide, such as chloride.

In an aspect, the transition metal complex can be an osmium transition metal complex that can comprise one or more ligands, wherein at least one (e.g., 1, 2, 3, 4, 5, or 6) ligand that can comprise a nitrogen-containing heterocycle (e.g., imidazole, pyrazole, oxazole, thiazole, pyridine, and pyrazine). In some aspects, the osmium transition metal complex can comprise one or more ligands selected from 4,4′-dimethyl-2,2′-bipyridine, mono-, di-, or polyalkoxy-2,2′-bipyridines (e.g., 4,4′-dimethoxy-2,2′-bipyridine), 4,7-dimethyl-1,10-phenanthroline, mono, di-, or polyalkoxy-1,10-phenanthrolines (e.g., 4,7-dimethoxy-1,10-phenanthroline).

In an aspect, the redox mediator can comprise an osmium complex bonded to a polymer or copolymer of poly(1-vinyl imidazole) or poly(4-vinylpyridine). The poly(4-vinylpyridine)-based polymer is a prepolymer that has been modified, as shown in the following structure, to attach an osmium complex (e.g., a poly(biimidizyl) osmium complex):

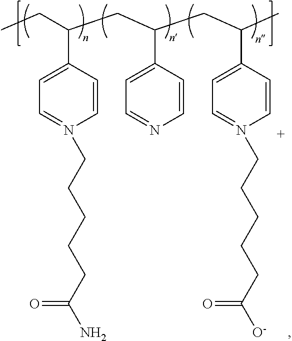

wherein n can be 2, n′ can be 17, and n″ can be 1. Other reactive groups and/or spacer groups can be used.

In an aspect, the electron redox mediator can comprise an osmium-containing poly(4-vinylpyridine)-based polymer, as shown below.

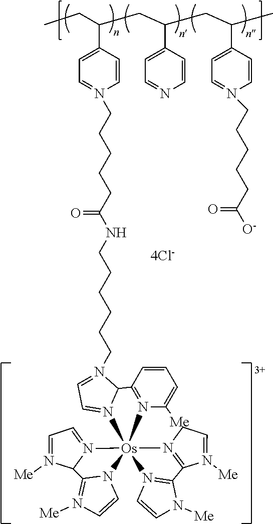

wherein n is 2, n′ is 17, and n″ is 1.

In some aspects, the electron transfer agent can be attached (e.g., non-leachably and/or covalently bonded) to the polymer in the redox mediator. For example, covalent bonding of the electron transfer agent to the polymer can take place by polymerizing a monomer unit bearing a covalently bound electron transfer agent, or the electron transfer agent can be reacted with the polymer separately after the polymer has already been synthesized.

According to some aspects, a bifunctional spacer can be used to attach (e.g., covalently bond) the electron transfer agent to the polymer in the redox mediator, with a first reactive group being reactive with the polymer (e.g., a functional group capable of quaternizing a pyridine nitrogen atom or an imidazole nitrogen atom) and a second reactive group being reactive with the electron transfer agent (e.g., a functional group that is reactive with a ligand coordinating a metal ion). Typically, covalent bonds are formed between the two reactive groups to generate a linkage. Suitable reactive groups include, for example, activated ester (e.g., succinimidyl, benzotriazolyl, or an aryl substituted with one more electron withdrawing groups, such as sulfo, nitro, cyano, or halo), acrylamido, acyl azido, acyl halide, carboxy (—COO— or —CO₂H), aldehyde, ketone, alkyl halide, alkyl sulfonato, anhydride, aziridino, epoxy, halotriazinyl, imido ester, isocyanato, isothiocyanato, maleimido, sulfonyl halide, amino, thiol (—SH), hydroxy, pyridinyl, imidazolyl, and hydroxyamino. The reaction between two reactive groups can form a covalent linkage between the transition metal complex and the polymer that is a carboxamido, thioether, hydrazonyl, oximyl, alkyamino, ester, carboxylic ester, imidazolium, pyridinium, ether, thioether, aminotriazinyl, triazinyl ether, amidinyl, urea, urethanyl, thiourea, thioether, sulfonamide, or any combination. In addition to the reactive groups, the bifunctional spacer typically can further comprise an alkylenyl (i.e., —(CH₂)ₙ—) and/or ethylenyloxy (i.e., —(CH₂CH₂O)ₘ—, in which n and m are each independently an integer from 1 to 12 (e.g., 1 to 11, 1 to 10, 1 to 9, 1 to 8, 1 to 7, 1 to 6, 1 to 5, 1 to 4, 1 to 3, or 1 to 2).

In some aspects, the redox mediator can further comprise a cross linking agent. In general, the cross linking agent is any suitable multifunctional (e.g., bifunctional) short chain molecule that enables the electron transfer agent to attach (e.g., covalently bond) to the polymer of the redox mediator. For example, the cross linking agent can be include a polyepoxide (e.g., a polyethylene glycol diglycidylether (PEGDGE), ethylene glycol diglycidyl ether (EGDGE), resorcinol diglycidyl ether, 1,2,7,8-diepoxyoctane, Gly3), cyanuric chloride, N-hydroxysuccinimide, an imidoester, epichlorohydrin, or a combination thereof. In an aspect, the cross linking agent is a polyethylene glycol diglycidylether (PEGDGE) of the following formula:

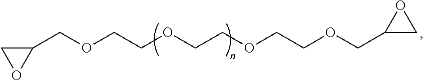

wherein n is an integer from 1 to about 50 (e.g., 1 to about 45, 1 to about 40, 1 to about 35, 1 to about 30, 1 to about 25, about 5 to about 50, about 5 to about 45, about 5 to about 40, about 5 to about 35, or about 5 to about 30).

In a particular example, the PEGDGE can be PEGDGE200, PEGDGE400 (n is 10), PEGDGE500, PEGDGE600, PEGDGE1000, or PEGDGE2000, in which the number denotes the average molecular weight (Mₙ). In an aspect, the crosslinking agent can be PEGDGE400.

The redox mediator can be applied to a working electrode using any suitable technique, such as spray coating, painting, inkjet printing, stenciling, roller coating, dip coating, or any combination thereof. In some aspects, the redox mediator can be applied by dip coating at least a portion of the working electrode into a solution of the redox mediator. One application or multiple applications can be applied (e.g., 1, 2, 3, 4, 5, 6, 7, 8, 9, or 10 applications). In some aspects, the redox mediator can be applied in 1, 2, 3, or 4 applications (e.g., passes). In some aspects, the redox mediator can be applied in 1 or 2 applications (e.g., passes).

In some aspects, one or more of the enzymes in the ketone-sensing layer or background sensing layer can be attached (e.g., covalently attached or unleachably bound) to the polymer portion of the redox mediator in the first sensing electrode. In some aspects, one or more of the enzymes can be covalently bonded to the polymer portion of the redox mediator. Covalent bonding of one or more of the enzymes to the redox mediator (e.g., polymer) can take place via a crosslinking agent, as described herein, and a reactive site on the enzyme. Thus in such instances, an enzyme can be electronically “wired” to a working electrode through the redox mediator. In an aspect, a hydrogel can be formed upon crosslinking an enzyme and its wire on electrodes. In another aspect, at least a portion of an enzyme can diffuse into the polymer and/or hydrogel and become attached but not necessarily covalently bonded to the polymer.

In an aspect, a first sensing electrode, the second sensing electrode, or both can comprise a membrane that overcoats at least the ketone sensing layer and/or the background sensing layer and optionally other components. The overcoating forms an outer membrane that provides stability to the sensing reagents (e.g., the ketone-responsive enzyme, the redox mediator), mass-transport limitations, biocompatibility, and/or prevents electrode fouling. The membrane can optionally coat all or part of the working electrode and optionally any counter or reference electrode that can be present. In an aspect, the membrane coats (e.g., encapsulates) the entire sensing system (e.g., the sensor tail), including the first and second sensing electrodes with their respective sensing layers, and any counter electrodes, reference electrodes, and/or substrates that can be present.

The membrane can comprise one or more polymeric membrane materials with a physical structure that allows analyte flux to the sensing layer (i.e., the membrane is a mass transport limiting membrane). The composition of the membrane can vary (e.g., the degree of hydrophobicity and/or the degree of crosslinking) to promote a desired flux of ketone(s) to the sensing electrode, thereby providing a desired signal intensity and stability, as described further herein. In an aspect, the membrane can be permeable to at least one ketone that is to be measured.

The coating of the membrane over at least the ketone sensing layer, at least the background sensing layer, or both can be performed by any suitable technique. Typically, the membrane can be coated by spray coating, painting, inkjet printing, roller coating, dip coating, or any combination thereof. The coating step can be performed once or multiple times (e.g., 2, 3, 4, 5, 6, 7, 8, 9, or 10 times), which will affect the thickness of the membrane coating. In an aspect, the coating step can be performed twice to form a bilayer.

In general, if multiple coats are applied, the first coat will be dried prior to applying the subsequent coat(s). The amount of time between coating steps will vary depending on the types of membrane, working electrode, and sensing layer, and the atmospheric conditions. In general, the drying time can be 1 minute or longer (e.g., 2 min or more, 3 min or more, 5 min or more, 10 min or more, 15 min or more, or 20 min or more). Once the membrane coating has been applied, the coating can be cured. In an aspect, the coating can be cured for 12 hours or more (e.g., 18 hours or more, 24 hours or more, 30 hours or more, 36 hours or more, 42 hours or more, or 48 hours or more). The curing can be at room temperature (i.e., about 20° C.) or at a slightly elevated temperature (e.g., 100° C. or less, 80° C. or less, 70° C. or less, 60° C. or less, 50° C. or less, 40° C. or less, 30° C. or less, or 25° C. or less). In general, the curing will not occur at less than about 20° C.

The membrane typically has a thickness that ranges from about 1 μm to about 100 μm. For example, in some aspects, the membrane can have a thickness of about 1 μm or more (e.g., about 5 μm or more, about 10 μm or more, about 15 μm or more, about 20 μm or more, about 25 μm or more, about 30 μm or more, about 35 μm or more, about 40 μm or more, about 50 μm or more, about 60 μm or more, about 70 μm or more, about 80 μm or more, or about 90 μm or more) and typically will have a thickness of about 100 μm or less (e.g., about 90 μm or less, about 80 μm or less, about 70 μm or less, about 60 μm or less, about 50 μm or less, about 45 μm or less, about 40 μm or less, about 35 μm or less, about 30 μm or less, about 25 μm or less, about 20 μm or less, about 15 μm or less, about 10 μm or less, or about 5 μm or less). In an example, the membrane can have a thickness of about 5 to about 85 μm, about 10 to about 65 μm, about 20 to about 50 μm, about 20 to about 40 μm, about 25 to about 35 μm, or a thickness of about 30 μm.

In an aspect, the membrane can comprise optionally crosslinked poly(4-vinylpyridine), poly(vinyl alcohol), poly(acrylic acid), poly(methacrylic acid), a tetrafluoroethylene-perfluoro-3,6-dioxa-4-methyl-7-octenesulfonic acid copolymer-based membrane (e.g., a NAFION™ membrane), polyurethane, or a combination thereof. In some aspects, the mass transport limiting membrane can comprise at least a poly(4-vinylpyridine) homopolymer or copolymer (e.g., poly(4-vinylpyridine)-co-polystyrenesulfonate (PVP-co-PSS)), in which the poly(4-vinylpyridine) can be optionally cross linked.

Suitable poly(4-vinylpyridine) copolymers for inclusion in the mass transport limiting membrane can comprise up to about 25% comonomers (based on the total amount of monomers in the copolymer), such as from about 0.1% to about 5% comonomers, or about 5% to about 15% comonomers, or about 15% to about 25% comonomers, or about 1% to about 10% co-monomers. Suitable comonomers are not particularly limited, provided that the mass transport limiting membrane affords sufficient ketone permeability to provide an analyte sensitivity of about 1 nA/mM or greater when exposed to ketone. In some aspects, the mass transport limiting membrane can comprise poly(4-vinylpyridine)-co-polystyrenesulfonate (PVP-co-PSS)) that is optionally crosslinked.

In some aspects, the membrane can comprise multiple layers in which each layer has a different composition and/or degree of crosslinking. In an example, the membrane coating can be a bilayer membrane that can comprise a first layer that can comprise a poly(4-vinylpyridine) homopolymer or copolymer and a second layer that can comprise a crosslinked poly(4-vinylpyridine) homopolymer or copolymer (e.g., crosslinked with PEGDGE). In some aspects, the membrane can comprise a bilayer of (i) a homopolymer of poly(4-vinylpyridine) crosslinked with a crosslinking agent and (ii) a copolymer poly(4-vinylpyridine) optionally crosslinked with a crosslinking agent, as described herein (e.g., high molecular weight (such as molecular weight 400 g/mol) poly(ethylene glycol) diglycidyl ether). In some aspects, the second layer can comprise a crosslinked polyvinylpyridine-co-styrene polymer. In some aspects, the second layer can comprise a polyvinylpyridine-co-styrene polymer, in which a portion of the pyridine nitrogen atoms are functionalized with a non-crosslinked poly(ethylene glycol) tail and a portion of the pyridine nitrogen atoms are functionalized with an alkylsulfonic acid group.

The coating of the membrane over at least the ketone sensing layer and/or the background sensing layer can be performed using any suitable technique. In some aspects, the membrane can be coated by spray coating, painting, inkjet printing, roller coating, dip coating, or any combination thereof. In an aspect, the coating comprises dipping the ketone sensor comprising the ketone sensing layer and the background sensing layer (e.g., the sensor tail) into a solution comprising the polymer and a solvent to provide a dipped ketone sensor. The coating step can be performed once or multiple times (e.g., 2, 3, 4, 5, 6, 7, 8, 9, or 10 times), which will affect the thickness of the membrane coating. In an aspect, the coating step can be performed twice to form a bilayer. In an aspect, the coating step can be a dip coating. In an aspect, the coating step can be a dip coating performed 2 to 6 (i.e., 2, 3, 4, 5, or 6) times.

The membrane typically has a thickness that ranges from about 1 μm to about 100 μm. For example, in some aspects, the membrane can have a thickness of about 1 μm or more (e.g., about 5 μm or more, about 10 μm or more, about 15 μm or more, about 20 μm or more, about 25 μm or more, about 30 μm or more, about 35 μm or more, about 40 μm or more, about 50 μm or more, about 60 μm or more, about 70 μm or more, about 80 μm or more, or about 90 μm or more) and typically will have a thickness of about 100 μm or less (e.g., about 90 μm or less, about 80 μm or less, about 70 μm or less, about 60 μm or less, about 50 μm or less, about 45 μm or less, about 40 μm or less, about 35 μm or less, about 30 μm or less, about 25 μm or less, about 20 μm or less, about 15 μm or less, about 10 μm or less, or about 5 μm or less). In an example, the membrane can have a thickness of about 5 to about 80 μm, about 10 to about 60 μm, about 20 to about 50 μm, about 20 to about 40 μm, about 25 to about 35 μm, or a thickness of about 30 μm.

In some aspects, the first and second electrodes are part of a sensor (e.g., a ketone sensor) that can be contained within a sensor housing that is configured for adherence to tissue (e.g., skin). If necessary, the sensor housing can include an adhesive layer that enables adhesion to a desired tissue. The sensor housing can hold all necessary components of the sensor, such as circuitry and a power source for operating the sensor. In some aspects, the power source (e.g., a coin cell battery) and/or active circuitry are not contained within the sensor housing. A processor can be communicatively coupled to the sensor, in which the processor is physically located within the sensor housing or a reader device. The power source can include one or more batteries, which can be rechargeable or single-use disposable batteries. Power management circuitry can regulate battery charging and power supply monitoring, boost power, or perform direct current (DC) conversions.

In some aspects, the sensor can comprise a sensor tail (e.g., insertion tip) configured for penetrating (e.g., implantation into) tissue. The sensor tail can comprise at least the first sensing electrode and the second sensing electrode. A counter electrode can be present in combination with one or both working electrodes. The various electrodes can be at least partially stacked (layered) upon one another and/or laterally spaced apart from one another upon the sensor tail. In general, the sensor tail can be of sufficient size and shape to be positionable below the surface of the tissue (e.g., penetrating through the skin (dermis)) and into the subcutaneous space and in contact with the wearer's biofluid, such as interstitial fluid. Suitable sensor configurations can be substantially flat in shape, substantially cylindrical in shape, or any other suitable shape. In an example, a sensor tail can be about 5 mm in length, about 0.6 mm in width, and about 0.25 mm in thickness. Suitable tissues include, for example, skin, including the dermal layer, an interstitial layer, and/or a subcutaneous layer of the skin. In any of the sensor configurations disclosed herein, the various electrodes can be electrically isolated from one another by a dielectric material or similar insulator.

In some aspects, the sensor can comprise a reference electrode, a counter electrode, or both a reference electrode and a counter electrode as part of the first sensing electrode, the second sensing electrode, or both. In an aspect, the counter electrode can be carbon (e.g., screen-printed carbon), and the reference electrode can be Ag/AgCl. In some aspects, a working electrode and a second electrode that functions as both a counter electrode and reference electrode (i.e., a counter/reference electrode) can be used as part of the first sensing electrode, the second sensing electrode, or both.

In an example, electrode contacts can be positioned on a first portion of the sensor situated above the skin surface and extend to a location in sensor tail. A first working electrode, a reference electrode, and a counter electrode can be at a second portion of the sensor and a second working electrode, reference electrode, and counter electrode can be at a third portion of the sensor, in which the second and third portions typically can be at a bottom portion of the sensor tail. The first working electrode can comprise a ketone sensing layer, and the second working electrode can comprise a background sensing layer, each as described herein.

In some aspects, the sensor can comprise at least one insulation (e.g., dielectric) layer as part of the first sensing electrode, the second sensing electrode, or both. In some aspects, the insulation layer can be comprised of a suitable dielectric material that can form a solid. In an example, the insulation layer can be formed from porcelain (ceramic), mica, glass, barium strontium titanate, a plastic (e.g., polystyrene, polytetrafluoroethylene, polyethylene terephthalate, polyethylene, polypropylene, polymethylmethacrylate, polysulfone, polydimethylsiloxane, polyvinyl chloride, or a combination thereof), or a metal oxide (e.g., silica, alumina, titania, zirconia, tantalum oxide, etc.).

In some aspects, the sensor can comprise a substrate, wherein the first and second sensing electrodes can be disposed on the substrate. The substrate can be formed from any suitable inert material. In some aspects, the substrate can be biocompatible. Examples of a suitable substrate include titanium, a carbon-based substrate (e.g., cellulose, polylactic acid) and a plastic substrate (e.g., polyethylene terephthalate, polyethylene, polypropylene, polymethylmethacrylate, polysulfone, polydimethylsiloxane, polyvinyl chloride, etc.). In some aspects, the substrate can be disposed between a working electrode and a counter and/or reference electrode as part of the first sensing electrode, the second sensing electrode, or both.

The sensor can be part of a system that can comprise a first sensing electrode (e.g., a ketone sensing electrode), a second sensing electrode (e.g., a background sensing electrode), and a circuit configured to connect and disconnect with first and second sensing electrodes. In an aspect, the system can be a ketone sensor comprising a first sensing electrode comprising a first working electrode, a ketone sensing layer, and a redox mediator; a second sensing electrode as the background sensing layer comprising a second working electrode, the redox mediator, and no ketone-responsive enzyme; and a membrane comprising PVP that overcoats at least the ketone sensing layer and background sensing layer. The ketone sensing layer can comprise 3-HBDH, diaphorase, nicotinamide adenine dinucleotide phosphate (NAD(P)+) or a derivative thereof, optionally an albumin, and an osmium-containing poly(4-vinylpyridine)-based polymer as the redox mediator. The background sensing layer can comprise diaphorase, nicotinamide adenine dinucleotide phosphate (NAD(P)+) or a derivative thereof, an albumin, and an osmium-containing poly(4-vinylpyridine)-based polymer as the redox mediator.

In the presence of a biofluid comprising an analyte, the first sensing electrode (ketone sensing electrode) oxidizes the analyte, and the amount of oxidation is measured as the amount of electron charge produced from the reaction. The second sensing electrode (background sensing electrode) oxidizes background interferents (e.g., electroactive interferents) present in the biofluid, and the amount of oxidation is measured as the amount of electron charge produced from the reaction. As long as the first and second sensing electrodes are not connected to another electrode, the charge from the redox reaction will continue to accumulate on the respective electrode. Accumulating charge (electrons) for a set period of time allows for low concentrations of ketone and background to result in a signal output that is easy to measure and quantify compared to other known methods. After a set period of time for charge accumulation, the first and/or second sensing electrode can be connected with at least one (e.g., 1, 2, 3, or 4) other electrode such as a counter electrode and/or reference electrode to form a circuit. Upon connecting the circuit, the accumulated electrons on the first and/or second sensing electrode can be discharged as an electrical signal, the amplitude of which can be measured and correlated to the amount of ketone present at the sensing electrode. Subtracting the measured background signal (i.e., a measure of background signal only) from the measured ketone signal (i.e., a measure of ketone signal+background signal) can provide the ketone only signal, which is proportional to the ketone concentration.

The sensing of the analyte (A) relies on having an oxidoreductase enzyme (AOx) electrically “wired” to the working electrode of the sensor through a redox mediator. During normal amperometric sensing, the electrode is poised at a potential (voltage) so that the analyte is reacted at a constant rate, which is proportional to the analyte concentration. For an analyte oxidation reaction (A to A′), the electrons will flow from the analyte (A) to the analyte-specific enzyme (AOx) to the redox mediator (e.g., Os³⁺) to the working electrode at a constant rate, producing a steady-state current. If the working electrode is disconnected from the circuit, the flow of electrons from the redox polymer to the working electrode will stop, resulting in no current flow through the circuit. However, the analyte will still undergo enzymatic oxidation, which in turn results in reducing the redox mediator (e.g., Os³⁺ to Os²⁺). This results in a buildup of the reduced form of the redox mediator (e.g., Os²⁺) over time, as electrons (e) from the analyte are stored in the redox mediator. When the working electrode is reconnected to the circuit so that it is poised at its original potential (voltage), the buildup of the reduced form of the redox mediator will be oxidized, resulting in a large current spike. The current will then decay back to the original amperometric current as the redox system reaches steady-state once again. This two-step process forms the basis for accumulation mode sensing: one in which the working electrode of the sensor is disconnected from or not connected to the circuit for a set period of time (also referred to as the accumulation time), enabling charge from the analyte to accumulate in the redox polymer, and a second in which the working electrode of the sensor is connected to the circuit after the accumulation time, enabling the accumulated charge to be discharged and measured as a sharp peak.

In some aspects, in a ketone sensor used herein, the potential (voltage) sufficient to drive the redox reaction and reduce background interference can be less than +40 mV (e.g., less than +30 mV, less than +20 mV, less than +10 mV, less than +5 mV, less than 0 mV, less than −5 mV, less than −10 mV, less than −20 mV, less than −30 mV, less than −40 mV, less than −50 mV, less than −60 mV, less than −70 mV, less than −80 mV, less than −90 mV, less than −100 mV, less than −110 mV, less than −120 mV, less than −130 mV, less than −140 mV, less than −150 mV, less than −160 mV, less than −170 mV, less than −180 mV, less than −190 mV, less than −200 mV, less than −210 mV, less than −220 mV, less than −230 mV, or less than −240 mV) versus Ag/AgCl.

In some aspects, the ketone signal and the background signal can be measured at different times. In some aspects, the ketone signal and the background signal can be obtained simultaneously via a first channel and a second channel.

Since ketone is present in a relatively low concentration in biofluid (e.g., serum), the system is designed to detect a low concentration of analyte by allowing for an accumulation of the analyte on an enzymatic biosensor. In the context of detecting ketone, a low concentration can be about 1 mM or less (e.g., about 900 μM or less, about 800 μM or less, about 700 μM or less, about 600 μM or less, about 500 μM or less, about 400 μM or less, about 300 μM or less, about 200 μM or less, or about 100 μM or less) to about 10 μM or more (e.g., about 20 μM or more, about 30 μM or more, about 40 μM or more, about 50 μM or more, about 60 μM or more, about 70 μM or more, about 80 μM or more, about 90 μM or more, about 100 μM or more, about 120 μM or more, about 140 μM or more, or about 150 μM or more). For example, the ketone concentration in an analyte can be about 10 μM to about 1 mM, about 50 to about 400 μM, about 60 to about 300 μM, or about 70 to about 200 μM.

In some aspects, the sensor is exposed to the biofluid in vivo. In general, the method uses a system (e.g., a ketone sensor), as disclosed herein, for measuring a concentration of ketone and can be used in an in vivo monitoring system, which while positioned in vivo in a user (e.g., a patient, such as a human) makes contact with the biofluid of the user and senses ketone contained therein. An in vivo monitoring system can include one or more reader devices that receives sensed analyte data from a sensor control device. The reader device can process and/or display the sensed analyte data or sensor data in any number of forms to the user. In some aspects, the reader device can be a mobile communication device, such as a dedicated reader device (configured for communication with a sensor control device) optionally in conjunction with a computer system, a mobile telephone (e.g., a WiFi or internet-enabled smart phone), a tablet, a personal digital assistant (PDA), or a mobile smart wearable electronics assembly (e.g., a smart glass, smart glasses, watch, bracelet, or necklace). Configuring a reader device to an in vivo monitoring system is described at, for example, U.S. Pat. No. 11,371,957, the disclosure of which is incorporated herein by reference in its entirety.

The reader device typically includes an input component, a display, and processing circuitry, which can include one or more processors, microprocessors, controllers, and/or microcontrollers, each of which can be a discrete chip or distributed amongst (and a portion of) a number of different chips. The processing circuitry can include a communications processor having on-board memory and an applications processor having on-board memory. The reader device can further include radio frequency (RF) communication circuitry coupled with an RF antenna, a memory, multi-functional circuitry with one or more associated antennas, a power supply, power management circuitry, and/or a clock. It will be recognized that other hardware and functionality can be included in the reader device.

In addition to the foregoing detection methods using a sensor (e.g., ketone sensor), the present disclosure relates to a ketone sensor comprising:
a first sensing electrode comprising a first working electrode and a ketone sensing layer on a portion of the first working electrode, wherein the ketone sensing layer comprises a ketone-responsive enzyme and a redox mediator; and
a second sensing electrode comprising a second working electrode and a background sensing layer on a portion of the second working electrode, wherein the background sensing layer comprises a redox mediator and no ketone-responsive enzyme.

The ketone-responsive enzyme is as described herein. In some aspects, the ketone-responsive enzyme can be 3-hydroxybutyrate dehydrogenase. In some aspects, the first sensing electrode can comprise an NAD(P)H oxidoreductase and nicotinamide adenine dinucleotide phosphate (NAD(P)+) or a derivative thereof. In some aspects, the first sensing electrode can comprise an albumin, as described herein. In some aspects, the first sensing electrode can comprise a pH buffer, as described herein.

In some aspects, the ketone-responsive enzyme can be attached to the redox mediator, as described herein.

In some aspects, the redox mediator can comprise a polymer and an electron transfer agent, each as described herein.

In some aspects, the polymer can comprise poly(vinylpyridine), poly(thiophene), poly(aniline), poly(pyrrole), or poly(acetylene). In some aspects, the polymer comprises a polymer or copolymer repeat unit comprising at least one pendant pyridinyl group, imidazolyl group, or both a pyridinyl and imidazolyl group. In some aspects, the polymer can be crosslinked with a cross linking agent, as described herein. In some aspects, the cross linking agent can be a polyepoxide, cyanuric chloride, N-hydroxysuccinimide, an imidoester, epichlorohydrin, or a combination thereof. In some aspects, the cross linking agent can be a polyethylene glycol diglycidylether (PEGDGE), as described herein.

In some aspects, the electron transfer agent can comprise a transition metal complex, as described herein. In some aspects, the transition metal complex can comprise osmium, ruthenium, iron, cobalt, or a combination thereof. In some aspects, the transition metal complex can be an osmium transition metal complex comprising one or more ligands, wherein at least one ligand comprises a nitrogen-containing heterocycle. In some aspects, the redox mediator can comprise an osmium complex bonded to a poly(vinylpyridine)-based polymer.

In some aspects, the ketone sensing layer or the background sensing layer can be continuous, or both sensing layers are continuous on the working electrode, as described herein. In some aspects, the ketone sensing layer or the background sensing layer can be discontinuous, or both sensing layers are discontinuous on the working electrode, as described herein.

In some aspects, the sensor can comprise a membrane, as described herein, overcoating at least the ketone sensing layer, at least the background sensing layer, or both. In some aspects, the membrane comprises poly(4-vinyl pyridine), which is optionally crosslinked.

In some aspects, the sensor can comprise a housing, as described herein. In some aspects, the sensor can comprise a sensor tail, as described herein, configured for implantation into a tissue, wherein the first and second sensing electrodes can be disposed on the sensor tail. In some aspects, the sensor can comprise a reference electrode, a counter electrode, or both a reference electrode and a counter electrode, each as described herein. In some aspects, the sensor can comprise at least one insulation layer, as described herein. In some aspects, the sensor can comprise at least one substrate, as described herein, wherein the first sensing electrode or the second sensing electrode is disposed on the substrate, or both sensing electrodes are disposed on the substrate.

In some aspects, the ketone sensor or a method of use thereof can provide an improved sensor accuracy by about 3-fold or higher (e.g., about 4-fold or higher, about 5-fold or higher, about 6-fold or higher, about 7-fold or higher, about 8-fold or higher, about 9-fold or higher, about 10-fold or higher, about 11-fold or higher, about 12-fold or higher, about 13-folder or higher, about 14-fold or higher, or about 15-fold or higher) compared to a ketone sensor that does not include one, two, or all three of the following features: a sensing potential less than +40 mV vs Ag/AgCl (e.g., a sensing potential at −80 mV), background subtraction, and accumulation mode sensing.

In some aspects, the ketone sensor or a method of use thereof can provide an increased signal by about 3-fold or higher (e.g., about 4-fold or higher, about 5-fold or higher, about 6-fold or higher, about 7-fold or higher, about 8-fold or higher, about 9-fold or higher, about 10-fold or higher, about 11-fold or higher, about 12-fold or higher, about 13-folder or higher, about 14-fold or higher, or about 15-fold or higher) compared to a ketone sensor that does not include one, two, or all three of the following features: a sensing potential less than +40 mV vs Ag/AgCl (e.g., a sensing potential at −80 mV), background subtraction, and accumulation mode sensing.

In some aspects, the ketone sensor can provide an accurate (e.g., within about 20% of actual, within about 18% of actual, within about 15% of actual, within about 12% of actual, within about 10% of actual, within about 8% of actual, within about 5% of actual, within about 4% of actual, within about 3% of actual, within about 2% of actual, or within about 1% of actual) ketone measurement. In some aspects, the ketone sensor can provide a ketone measurement within about 10% of actual (e.g., versus a control). In any of these aspects, the ketone sensor can provide an accurate ketone measurement over a period of one day or more (e.g., 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more, 16 days or more, 17 days or more, 18 days or more, 19 days or more, 20 days or more, or 21 days or more). In some aspects, the ketone sensor can provide an accurate ketone measurement over a period of 7 days or more. In some aspects, the ketone sensor can provide an accurate ketone measurement over a period of 14 days or more. In some aspects, the ketone sensor can provide an accurate ketone measurement over a period of 21 days or more.

### EXAMPLES

The examples presented below are provided for the purpose of illustration only and the aspects described herein should in no way be construed as being limited to these examples. Rather, the aspects should be construed to encompass any and all variations which become evident as a result of the teaching provided herein.

### Example 1

To determine the relationship between the sensing potential and background interference, a ketone sensor with a first sensing electrode and second sensing electrode were prepared in accordance with Tables 1 and 2. Membrane solutions were prepared in accordance with Table 3.

TABLE 1

| | Sensing Chemistry | Details |
|---|---|---|
| First | Standard | 120 mg/mL HBDH (0.1 mL) |
| sensing | (ketone sensing) | 60 mg/mL Diaphorase (0.1 mL) |
| electrode | | 120 mg/mL HSA (0.1 mL) |
| | | 120 mg/mL NAD (0.1 mL) |
| | | 60 mg/mL Os-PVP (0.1 mL) |
| | | 60 mg/mL PEG400DGE (0.1 mL) |
| | | 10 mM MES, pH 5.5 (0.3 mL) |
| Second | Blank | 60 mg/mL Diaphorase (0.1 mL) |
| sensing | (background sensing) | 120 mg/mL HSA (0.2 mL) |
| electrode | | 120 mg/mL NAD (0.1 mL) |
| | | 60 mg/mL Os-PVP (0.1 mL) |
| | | 60 mg/mL PEG400DGE (0.1 mL) |
| | | 10 mM MES, pH 5.5 (0.3 mL) |

HBDH: hydroxybutyrate dehydrogenase
HSA: human serum albumin
NAD: nicotinamide adenine dinucleotide
Os-PVP: osmium-containing poly(4-vinylpyridine)-based polymer
PEGDGE400: polyethylene glycol diglycidylether with an average molecular weight (Mn) of about 400 g/mol
MES: 2-(N-morpholino)ethanesulfonic acid

TABLE 2

| Group | Sensing chemistry | Dip-coating |
|---|---|---|
| Blank | Blank | PVP: 5 dips × 5 mm/s |
| | 2-pass (3 × 18 dispense pattern) | PVPS: 6 dips × 15 mm/s |
| Standard | Standard | |
| | 2-pass (3 × 18 dispense pattern) | |

PVP: poly(4-vinylpyridine) (PVP)
PVPS: crosslinked polyvinylpyridine-co-styrene polymer

TABLE 3

| Membrane | Details |
|---|---|
| Membrane 1 | 100 mg/mL PVP washed (5 mL) |
| | 100 mg/mL PEG400DGE (0.25 mL) |
| | 100 mg/mL PDMS (0.0165 mL) |
| | in 95:5 EtOH:water |
| Membrane 2 | 80 mg/mL PVPS (4 mL) |
| | 12.5 mg/mL Gly-3 (1 mL) |
| | 100 mg/mL PDMS (0.0165 mL) |
| | in 95:5 EtOH:water |

PVP: poly(4-vinylpyridine) (PVP)
PEGDGE400: polyethylene glycol diglycidylether with an average molecular weight (Mn) of about 400 g/mol
PDMS: po10qly(dimethylsiloxane)
EtOH: ethanol
PVPS: crosslinked polyvinylpyridine-co-styrene polymer
Gly-3: Gly-Gly-Gly, where Gly is glycine

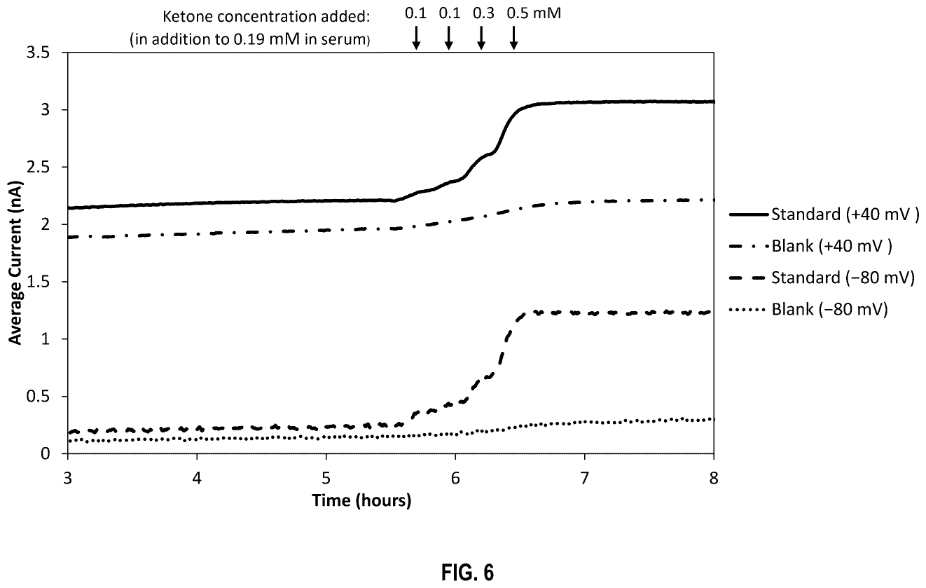

Sensors were tested in single-donor serum with a measured ketone (D-β-hydroxybutyrate) concentration of 190 μM using standard amperometry at either +40 mV or −80 mV vs Ag/AgCl. Aliquots of ketone (+100 μM, +100 μM, +300 μM, and +500 μM D-β-hydroxybutyrate, respectively) were cumulatively added to the serum every 15 minutes. The total ketone (D-β-hydroxybutyrate) concentration was 290 μM after the 1st aliquot of 100 μM added, 390 μM after the 2nd aliquot of 100 μM added, 690 μM after the 3rd aliquot of 300 μM added, and 1,190 μM after the 4th aliquot of 500 μM added. Time (hours) and current were recorded at a 5 minute data acquisition (DAQ) rate using a multichannel potentiostat (FIG. 6).

As seen in FIG. 6, it was observed that the Standard sensing chemistry showed a higher signal than the Blank sensing chemistry as a result of signal from ketone in the serum. However, the Blank sensing chemistry showed much lower background at −80 mV compared to +40 mV, indicating that the decreased sensing potential significantly reduced background signal. In turn, this allowed for more accurate ketone measurement.

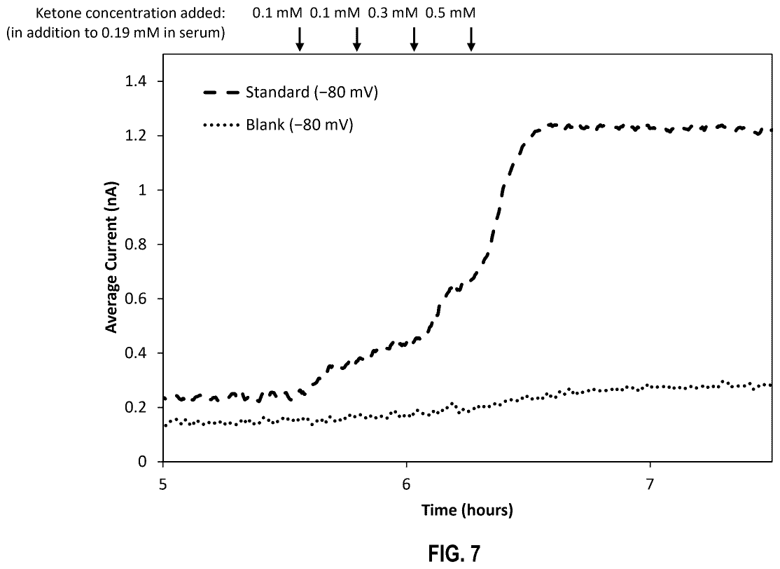

FIG. 7 shows a focused view of the added ketone aliquots of FIG. 6. As seen in FIG. 7, the initial Standard sensor signal represents the sum of signal from ketone present in the serum and background interference from the serum. The Blank sensor signal represents the serum background interference alone. The Blank signal can be subtracted from the Standard signal to determine signal from ketone alone. However, because the ketone concentration is very low, this difference cannot be determined accurately due to noise related to electronics. This background noise will be more extreme for a sensor being worn.

### Example 2

To determine the effect of accumulation mode sensing, the same sensor from Example 1 was used. In the cumulative detection mode, the Blank and Ketone electrodes were left unpoised/open-circuit for 2.5 minutes and then connected at −80 mV for 2.5 minutes. This cycle was repeated continuously as aliquots of ketone (+100 μM, +100 μM, +300 μM, and +500 μM D-β-hydroxybutyrate, respectively) were cumulatively added to the serum every 30 minutes. The total ketone (D-β-hydroxybutyrate) concentration was 290 μM after the 1st aliquot of 100 μM added, 390 μM after the 2nd aliquot of 100 μM added, 690 μM after the 3rd aliquot of 300 μM added, and 1,190 μM after the 4th aliquot of 500 μM added.

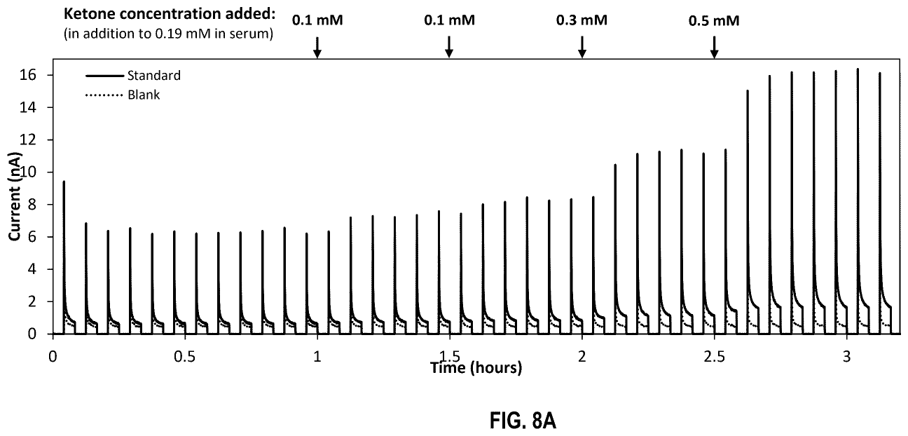

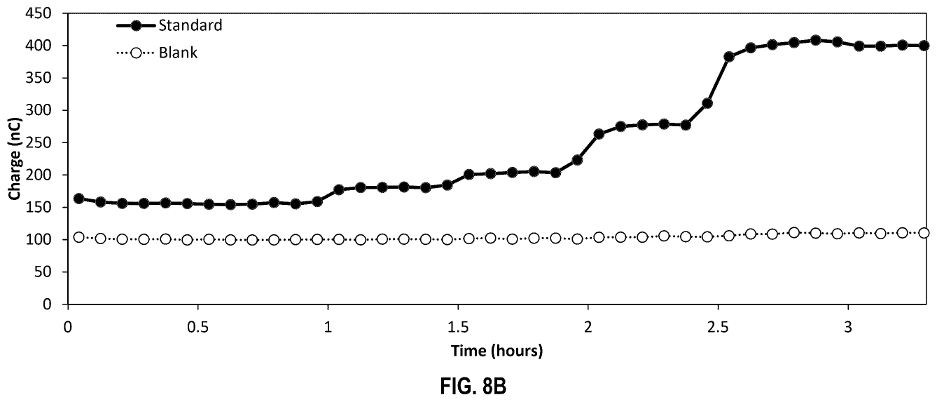

FIG. 8A shows current (nA) versus time (hours). FIG. 8B shows the integrated charge for each peak. As seen in FIG. 8A, the peak heights are >5 nA, which are much higher than the 0.1 to 0.3 nA observed in the previous experiment with standard amperometry (FIG. 6). This demonstrates that cumulative detection increases sub-mM ketone signal to levels similar to other analytes, such as glucose sensors, by providing acceptable signal-to-noise ratios.

Based on these measurements, the ketone concentration was measured, as set forth in Table 4.

TABLE 4
Baseline signal (serum with 190 μM ketone)
Blank baseline: 100 ± 3 nC
Standard baseline: 155 ± 5 nC
Ketone response (determined from change
in signal upon ketone additions)
Standard response: 247 ± 18 nC/mM

| | |
|---|---|
| Without | Predicted [ketone] = Standard Baseline/Standard response = |
| background | (155 nC)/(247 nC/mM) = |
| subtraction | 0.63 mM |
| With | Predicted [ketone] = Standard Baseline − Blank Baseline/ |
| background | Standard response = |
| subtraction | (155 nC − 100 nC)/(247 nC/mM) = |
| | 0.22 mM |
| Serum | Actual [ketone] according to serum panel = 0.19 mM |
| clinical lab | |
| results | |

As seen in Table 4, the predicted ketone without background subtraction dramatically overestimated the serum ketone concentration (about 232% overestimation) while the value using background was significantly more accurate (about 16% overestimation).

### Example 3

Two ketone sensors (“Sensor 1” and “Sensor 2”) each with a first sensing electrode and a second sensing electrode, were prepared in accordance with Tables 5 and 6, to test whether low-concentration ketone monitoring in vivo was enhanced by reduced sensing potential, background subtraction, and accumulation mode. As shown in Tables 5 and 6, the sensing chemistry was deposited on the sensing electrodes with a non-contact piezoelectric dispense system in a pattern that is 5 drops wide by 10 drops tall (first pass). Each drop was about 0.6 nL and the drops join together to form a connected “slot” pattern. The sensing chemistry was dried and then the sensing chemistry was dispensed in a 5×10 pattern again (second pass).

Membrane solutions were prepared in accordance with Table 7. A membrane was deposited on the working electrodes by dip-coating the electrodes 5 times at a speed of 5 mm/s in the membrane solution prepared. The electrodes were then baked at 56° C. for 3 days to form the ketone sensor.

TABLE 5

| | Sensing Chemistry | Details |
|---|---|---|
| First | Standard | 120 mg/mL HBDH (0.1 mL) |
| sensing | (ketone sensing) | 60 mg/mL Diaphorase (0.1 mL) |
| electrode | | 120 mg/mL HSA (0.1 mL) |
| | | 120 mg/mL NAD (0.1 mL) |
| | | 60 mg/mL Os-PVP (0.1 mL) |
| | | 60 mg/mL PEG400DGE (0.1 mL) |
| | | 10 mM MES, pH 5.5 (0.3 mL) |
| Second | Blank | 60 mg/mL Diaphorase (0.1 mL) |
| sensing | (background sensing) | 120 mg/mL HSA (0.2 mL) |
| electrode | | 120 mg/mL NAD (0.1 mL) |
| | | 60 mg/mL Os-PVP (0.1 mL) |
| | | 60 mg/mL PEG400DGE (0.1 mL) |
| | | 10 mM MES, pH 5.5 (0.3 mL) |

TABLE 6

| | |
|---|---|
| Sensing Chemistry: | Working Electrode 1: standard (ketone sensing) |
| | 2-pass (5 × 10 dispense pattern) |
| | Working Electrode 2: blank (background sensing) |
| | 2-pass (5 × 10 dispense pattern) |
| Sensor Tail | 0.4 mm wide, 6.6 mm long |
| Dimensions by | |
| Laser-Cutting: | |
| Dip-coating: | PVP 5 dips × 5 mm/s |
| Baking: | 56° C., 3 days |

TABLE 7

| Membrane | Details |
|---|---|
| PVP | 100 mg/mL PVP washed (5 mL) |
| | 100 mg/mL PEG400DGE (0.25 mL) |
| | 100 mg/mL PDMS (0.0165 mL) |
| | in 95:5 EtOH:water |

Sensors 1 and 2 were placed on opposite arms of a subject and ketone concentration was continuously monitored over a 16 hour period starting at 4:00 PM. The subject drank an increasing amount (2 mL, 4 mL, 8 mL, 16 mL, and 32 ml) of a ketone drink (KETONE-IQ) every hour. Sensor 1 measured ketone concentration using cumulative detection (the Blank and Ketone electrodes were left unpoised/open-circuit for 2.5 minutes and then connected at −80 mV for 2.5 minutes). Sensor 2 measured ketone concentration using standard amperometry at −80 mV vs Ag/AgCl.

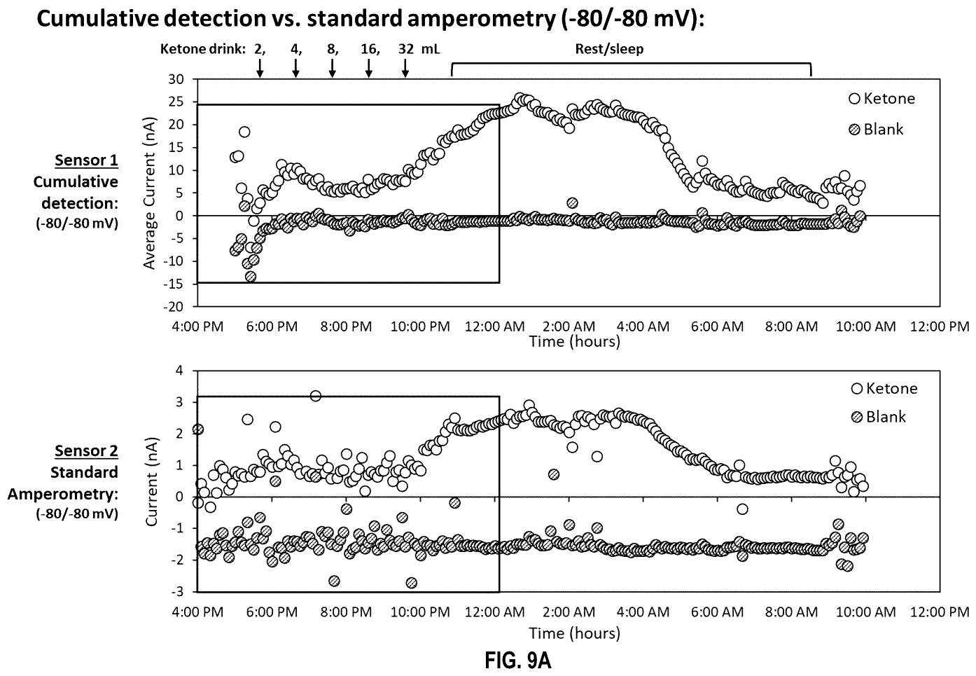

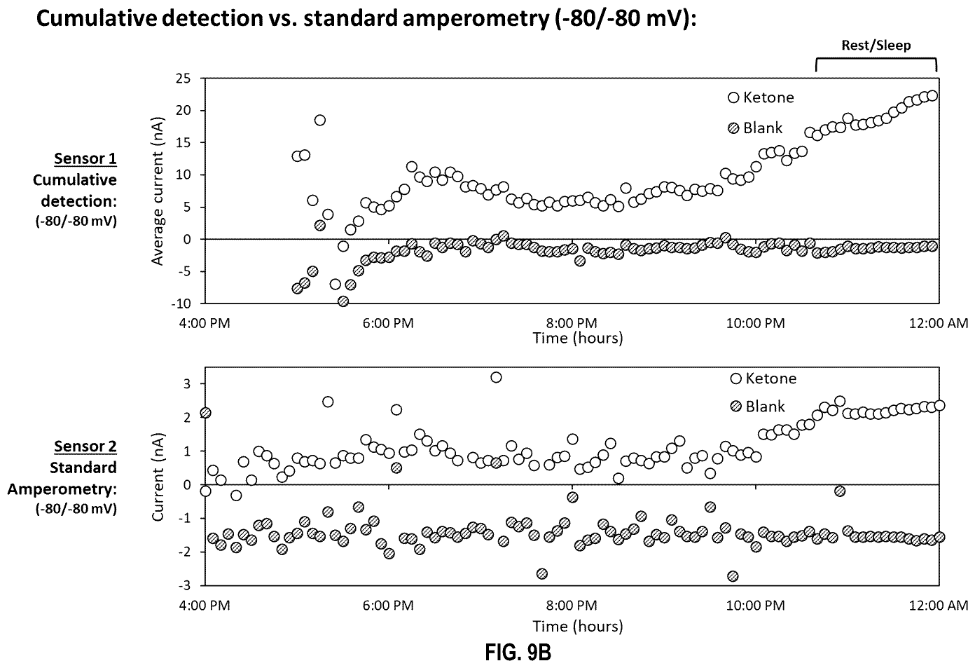

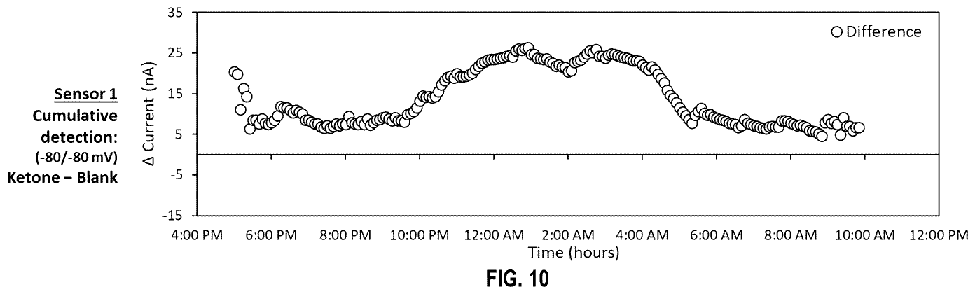

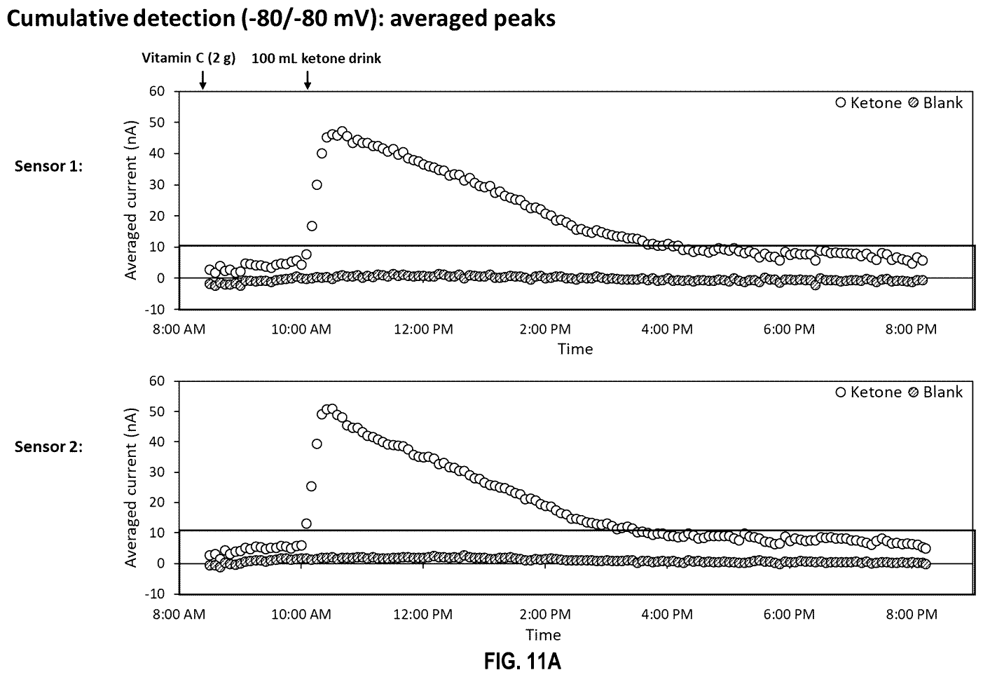

As seen in FIGS. 9A-9B, it was observed that cumulative detection (i.e. Sensor 1) increased signal about 10-fold and decreased noise relative to standard amperometry (Sensor 2). Signal-to-background ratio was also significantly higher in Sensor 1 than Sensor 2. And as seen in FIG. 10, background subtraction stabilized the cumulative detection signal, especially in noisy regions. The reduced sensing potential (−80 mV) also reduced background interference as seen in the relatively low signal from the background channel.

### Example 4

The consistency and sensitivity of ketone readings for ≤1 mM ketone concentrations were confirmed using blood ketone test strip measurements. Sensors 1 and 2 were placed on opposite arms of a subject and ketone concentration was continuously monitored. Sensors 1 and 2 measured ketone concentration using cumulative detection (the Blank and Ketone electrodes were left unpoised/open-circuit for 2.5 minutes and then connected at −80 mV for 2.5 minutes).

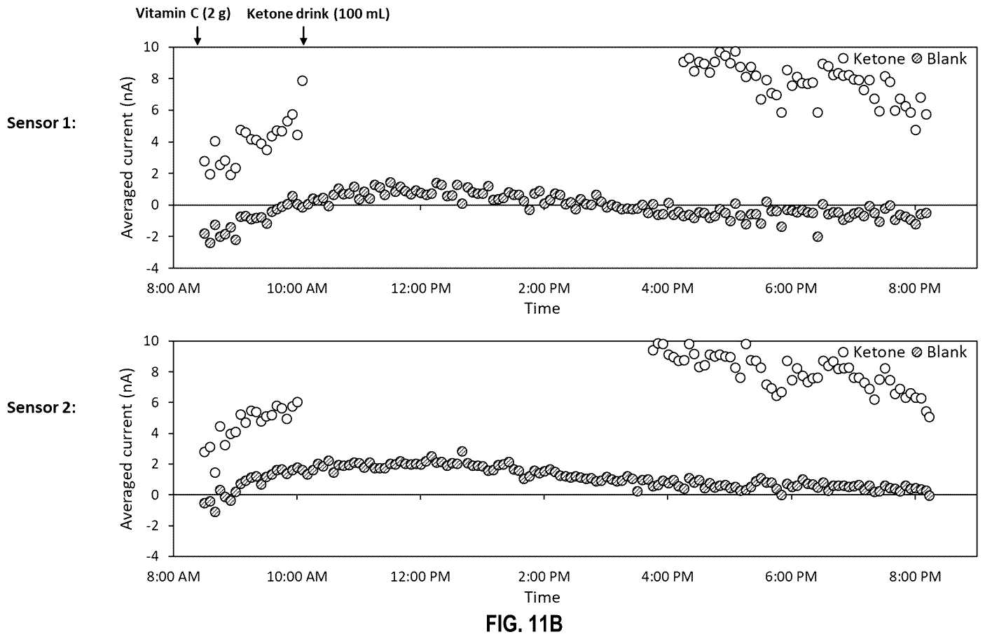

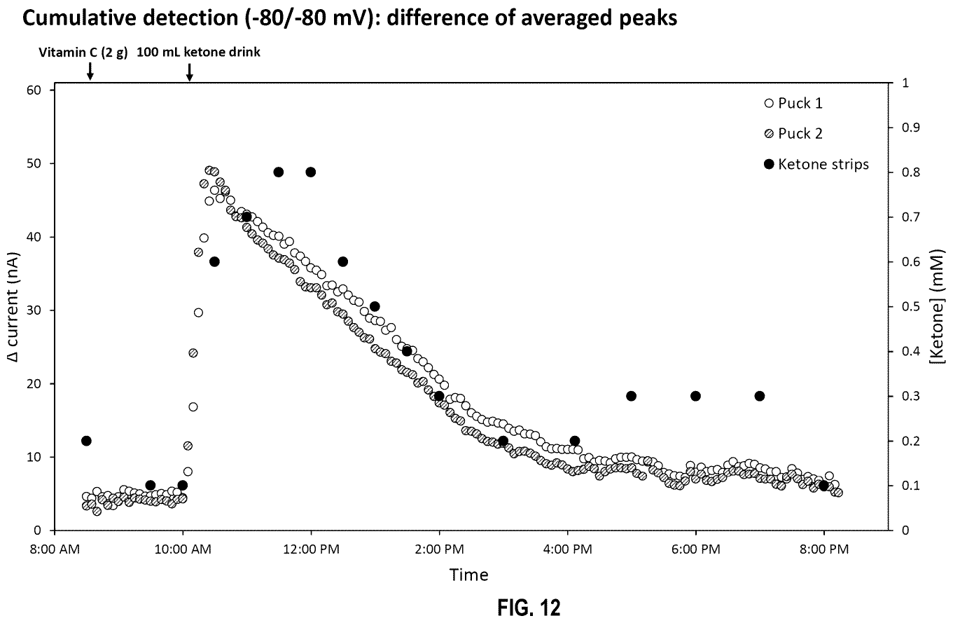

The subject was given Vitamin C (2 g) at 8:30 AM and then a ketone drink (100 mL) at 10:00 AM and ketone concentration was continuously monitored by Sensors 1 and 2 throughout the day. Reference measurements were also taken using blood ketone test strips. As shown in FIGS. 11A-11B, Sensors 1 and 2 showed high signal and low noise at ≤1 mM ketone concentrations. The background signal was less significant at −80 mV than +40 mV in Sensors 1 and 2, but the background channels did detect the Vitamin C (used as a background interferent). The signals from Sensors 1 and 2 after background subtraction plotted against the blood ketone strip measurements confirmed that Sensors 1 and 2 provided consistent and sensitive ketone readings at ≤1 mM ketone concentrations (FIG. 12).

It is to be appreciated that the Detailed Description section, and not the Summary and Abstract sections, is intended to be used to interpret the claims. The Summary and Abstract sections may set forth one or more but not all exemplary aspects of the present invention as contemplated by the inventor(s), and thus, are not intended to limit the present invention and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

1. A method for sensing ketone comprising:
contacting a biofluid comprising a ketone with
i) a first sensing electrode comprising a ketone-responsive enzyme and a redox mediator; and
ii) a second sensing electrode comprising a redox mediator and no ketone-responsive enzyme;
connecting the first and second sensing electrodes to a circuit and applying a potential less than +40 mV to both electrodes to provide a steady state;
disconnecting the first and second sensing electrodes from the circuit;
accumulating a charge derived from the biofluid reacting with the first and second sensing electrodes for a set period of time;
connecting the first and second sensing electrodes to the circuit after the set period of time; and
measuring a ketone signal by subtracting a signal obtained from the second sensing electrode from a signal obtained from the first sensing electrode.

2. A method for sensing ketone comprising:
(a) contacting first and second sensing electrodes with a biofluid comprising a ketone, wherein the first sensing electrode comprises a ketone-responsive enzyme and a redox mediator and the second sensing electrode comprises a redox mediator and no ketone-responsive enzyme;
(b) connecting the first sensing electrode to a circuit and applying a potential less than +40 mV to provide a steady state;
(c) disconnecting the first sensing electrode from the circuit and connecting the second sensing electrode to the circuit and applying a potential less than +40 mV to provide a steady state;
(d) accumulating a charge derived from the biofluid reacting with the first sensing electrode for a first set period of time;
(e) connecting the first sensing electrode to the circuit after the first set period of time and disconnecting the second sensing electrode from the circuit;
(f) accumulating a charge derived from the biofluid reacting with the second sensing electrode for a second set period of time;
(g) connecting the second sensing electrode to the circuit after the second set period of time; and
(h) measuring a ketone signal by subtracting a signal obtained from the second sensing electrode from a signal obtained from the first sensing electrode.

3-32. (canceled)

33. A ketone sensing system, comprising:
(i) a ketone sensor comprising a first portion configured to be positioned above a user's skin and a second portion configured to be positioned below the user's skin and in contact with the user's biological fluid to monitor the level of ketone in vivo, the second portion comprising:
a) a first sensing electrode configured to generate a first signal, the first sensing electrode comprising a first working electrode and a ketone sensing layer on a portion of the first working electrode, wherein the ketone sensing layer comprises a ketone-responsive enzyme and a redox mediator;
b) a second sensing electrode configured to generate a background signal, the second sensing electrode comprising a second working electrode and a background sensing layer on a portion of the second working electrode, wherein the background sensing layer comprises a redox mediator and no ketone-responsive enzyme; and
c) a counter electrode or a reference electrode configured to connect to a circuit with the first and second sensing electrodes;
wherein the first and second sensing electrodes are configured to (i) connect to the circuit to provide a steady state current at a potential less than +40 mV, (ii) disconnect from the circuit such that a charge derived from the first and second sensing electrodes is accumulated for a set period of time, and (iii) reconnect to the circuit after the set period of time to generate the first signal and the background signal;
(ii) a processor configured to subtract the background signal generated from the second sensing electrode from the first signal obtained from the first sensing electrode to generate a ketone signal, wherein the ketone signal is proportional to a concentration of ketones.

34. The ketone sensor system of claim 33, wherein the ketone-responsive enzyme is 3-hydroxybutyrate dehydrogenase.

35. The ketone sensor system of claim 33, wherein the first sensing electrode further comprises an NAD(P)H oxidoreductase and nicotinamide adenine dinucleotide phosphate (NAD(P)+) or a derivative thereof.

36. The ketone sensor system of claim 33, wherein the ketone-responsive enzyme is attached to the redox mediator.

37. The ketone sensor system of claim 33, wherein the first sensing electrode further comprises an albumin.

38. The ketone sensor system of claim 33, wherein the first sensing electrode further comprises a pH buffer.

39. The ketone sensor system of claim 33, wherein the redox mediator comprises a polymer and an electron transfer agent.

40. The ketone sensor system of claim 39, wherein the polymer comprises poly(vinylpyridine), poly(thiophene), poly(aniline), poly(pyrrole), or poly(acetylene).

41. The ketone sensor system of claim 40, wherein the polymer comprises a polymer or copolymer repeat unit comprising at least one pendant pyridinyl group, imidazolyl group, or both a pyridinyl and imidazolyl group.

42. The ketone sensor system of claim 39, wherein the polymer is crosslinked with a crosslinking agent.

43. The ketone sensor system of claim 42, wherein the crosslinking agent is a polyepoxide, cyanuric chloride, N-hydroxysuccinimide, an imidoester, epichlorohydrin, or a combination thereof.

44. The ketone sensor system of claim 42, wherein the crosslinking agent is a polyethylene glycol diglycidylether (PEGDGE).

45. The ketone sensor system of claim 39, wherein the electron transfer agent comprises a transition metal complex.

46. The ketone sensor system of claim 45, wherein the transition metal complex comprises osmium, ruthenium, iron, cobalt, or a combination thereof.

47. The ketone sensor system of claim 45, wherein the transition metal complex is an osmium transition metal complex comprising one or more ligands, wherein at least one ligand comprises a nitrogen-containing heterocycle.

48. The ketone sensor system of claim 33, wherein the redox mediator comprises an osmium complex bonded to a poly(vinylpyridine)-based polymer.

49. The ketone sensor system of claim 33, wherein the ketone sensing layer or the background sensing layer is continuously disposed on the working electrode, or both sensing layers are continuously disposed on the working electrode.

50. The ketone sensor system of claim 33, wherein the ketone sensing layer or the background sensing layer is discontinuously disposed on the working electrode, or both sensing layers are discontinuously disposed on the working electrode.

51. The ketone sensor system of claim 33, further comprising a membrane overcoating at least the ketone sensing layer, at least the background sensing layer, or both.

52. The ketone sensor system of claim 51, wherein the membrane comprises poly(4-vinyl pyridine), which is optionally crosslinked.

53. (canceled)

54. (canceled)

55. The ketone sensor system of claim 3353 or 54, wherein the second portion comprises a counter electrode.

56. The ketone sensor system of claim 33, further comprising at least one insulation layer.

57. The ketone sensor system of claim 33, further comprising at least one substrate, wherein the first sensing electrode or the second sensing electrode is disposed on the substrate, or both sensing electrodes are disposed on the substrate.

58. The ketone sensing system of claim 33, wherein the first and second sensing electrodes are configured to alternate connecting to the circuit such that only one of the first and second sensing electrodes is connected to the circuit at a time.
